SEQUENCE LISTING (19 sequences)

SEQ ID NO: 1 - DNA, 3782 bp, synthetic construct
  source 1..3782: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagatgaggctcgtttagtgaaccgtcagatcgcctggagacgccatccacgctgttttgacctccatagaagacaccgggaccgatccagcctccggactctagaggatcgaacccttttggaccctcgtacagaagctaatacgactcactatagggaaataagagagaaaagaagagtaagaagaaatataagagccaccatggtgtctaagggcgaagagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatgctgccttctgcggggcttgccttctggccatgcccttcttctctcccttgcacctgtacctcttggtctttgaataaagcctgagtaggaagtgagggtctagaactagtgtcgacgcaaatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 3749 bp, synthetic construct
  source 1..3749: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagatgaggctcgtttagtgaaccgtcagatcgcctggagacgccatccacgctgttttgacctccatagaagacaccgggaccgatccagcctccggactctagaggatcgaacccttttggaccctcgtacagaagctaatacgactcactatagggaaataagagagaaaagaagagtaagaagaaatataagagccaccatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtaggctgccttctgcggggcttgccttctggccatgcccttcttctctcccttgcacctgtacctcttggtctttgaataaagcctgagtaggaagtgagggtctagaactagtgtcgacgcaaatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 6711 bp, synthetic construct
  source 1..6711: mol_type = other DNA
gatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctggaattcctcgaaagacaactcagagttcaccatgggctccatcggtgcagcaagcatggaattttgttttgatgtattcaaggagctcaaagtccaccatgccaatgagaacatcttctactgccccattgccatcatgtcagctctagccatggtatacctgggtgcaaaagacagcaccaggacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaagcatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttccccttaaaaagaagaaagctgaaaaactctgtcccttccaacaagacccagagcactgtagtatcaggggtaaaatgaaaagtatgttatctgctgcatccagacttcataaaagctggagcttaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattctttt

SEQ ID NO: 4 - DNA, 27 bp, synthetic construct
  source 1..27: mol_type = other DNA
ttggaccctcgtacagaagctaatacg

SEQ ID NO: 5 - DNA, 170 bp, synthetic construct
  source 1..170: mol_type = other DNA
aagcaagctgaccctgaagttttttttttttttttttttttttttttttttttttttttttttttttttttttttttttttttttttttttttttttttttttttttttttttttttttttttttttttttttttttttttcttcctactcaggctttattcaaagacca

SEQ ID NO: 6 - DNA, 21 bp, synthetic construct
  source 1..21: mol_type = other DNA
gcaagctgaccctgaagtttt

SEQ ID NO: 7 - DNA, 25 bp, synthetic construct
  source 1..25: mol_type = other DNA
gcaagctgaccctgaagtttttttt

SEQ ID NO: 8 - DNA, 170 bp, synthetic construct
  source 1..170: mol_type = other DNA
aaaacttcagggtcagcttgcttttttttttttttttttttttttttttttttttttttttttttttttttttttttttttttttttttttttttttttttttttttttttttttttttttttttttttttttttttttttcttcctactcaggctttattcaaagacca

SEQ ID NO: 9 - DNA, 21 bp, synthetic construct
  source 1..21: mol_type = other DNA
aacttcagggtcagcttgctt

SEQ ID NO: 10 - DNA, 25 bp, synthetic construct
  source 1..25: mol_type = other DNA
aacttcagggtcagcttgctttttt

SEQ ID NO: 11 - DNA, 20 bp, synthetic construct
  source 1..20: mol_type = other DNA
gagaacccactgcttactgg

SEQ ID NO: 12 - DNA, 163 bp, synthetic construct
  source 1..163: mol_type = other DNA
aaagtaggtgcaggaactgtgtttttttttttttttttttttttttttttttttttttttttttttttttttttttttttttttttttttttttttttttttttttttttttttttttttttttttttttttttttttttttcagcgagctctagcatttagg

SEQ ID NO: 13 - DNA, 21 bp, synthetic construct
  source 1..21: mol_type = other DNA
agtaggtgcaggaactgtgtt

SEQ ID NO: 14 - DNA, 25 bp, synthetic construct
  source 1..25: mol_type = other DNA
agtaggtgcaggaactgtgtttttt

SEQ ID NO: 15 - DNA, 163 bp, synthetic construct
  source 1..163: mol_type = other DNA
aacacagttcctgcacctacttttttttttttttttttttttttttttttttttttttttttttttttttttttttttttttttttttttttttttttttttttttttttttttttttttttttttttttttttttttttttcagcgagctctagcatttagg

SEQ ID NO: 16 - DNA, 21 bp, synthetic construct
  source 1..21: mol_type = other DNA
cacagttcctgcacctacttt

SEQ ID NO: 17 - DNA, 25 bp, synthetic construct
  source 1..25: mol_type = other DNA
cacagttcctgcacctacttttttt

SEQ ID NO: 18 - DNA, 195 bp, synthetic construct
  source 1..195: mol_type = other DNA
aagcaagctgaccctgaagttaaaaaagcaagctgaccctgaagttttttttttttttttttttttttttttttttttttttttttttttttttttttttttttttttttttttttttttttttttttttttttttttttttttttttttttttttttttttttttcttcctactcaggctttattcaaagacca

SEQ ID NO: 19 - DNA, 195 bp, synthetic construct
  source 1..195: mol_type = other DNA
aaaacttcagggtcagcttgcaaaaaaaacttcagggtcagcttgcttttttttttttttttttttttttttttttttttttttttttttttttttttttttttttttttttttttttttttttttttttttttttttttttttttttttttttttttttttttttcttcctactcaggctttattcaaagacca

(19) United States
(12) Patent Application Publication
(10) Pub. No.: US 2024/0327832 A1
(43) Pub. Date: Oct. 3, 2024

(54) NUCLEIC ACID CONSTRUCT ENABLING EXPRESSION AND INHIBITION OF GENE SIMULTANEOUSLY

(71) Applicants: INDUSTRY-ACADEMIC COOPERATION FOUNDATION GYEONGSANG NATIONAL UNIVERSITY, Jinju-si Gyeongsangnam-do (KR); EWHA UNIVERSITY - INDUSTRY COLLABORATION FOUNDATION, Seodaemun-gu Seoul (KR)

(72) Inventors: Kyuri LEE, Jinju-si Gyeongsangnam-do (KR); Hyuk Jin LEE, Seocho-gu Seoul (KR)

(73) Assignees: INDUSTRY-ACADEMIC COOPERATION FOUNDATION, Jinju-si Gyeongsangnam-do (KR); EWHA UNIVERSITY - INDUSTRY, Seodaemun-gu Seoul (KR)

(21) Appl. No.: 18/268,179

(22) PCT Filed: Dec. 16, 2020
(86) PCT No.: PCT/KR2020/018484

§ 371 Date: Apr. 19, 2024

(51) Int. Cl. C12N 15/113; A61K 39/00; A61P 35/00

(52) U.S. Cl. CPC C12N 15/113; A61K 39/0011; A61P 35/00; A61K 2039/53; C12N 2310/14; C12N 2830/50

(57) ABSTRACT

The present invention relates to a nucleic acid construct that can express and inhibit a gene simultaneously. The nucleic acid construct that can express and inhibit a gene simultaneously, according to the present invention, comprises both mRNA and siRNA in a single construct, and thus, mRNA and siRNA are introduced simultaneously into the same cell. Accordingly, a protein to be expressed may be expressed normally while expression of a particular gene may be specifically inhibited. The mRNA-siRNA nucleic acid construct activates an immune system by expressing a target protein in an antigen-presenting cell, and at the same time, overcomes interference of immune escape by blocking the activity of an immune checkpoint protein, and thus is expected to be remarkably useful in the fields of immunotherapy, especially in the field of cancer immunotherapy.

### RELATED APPLICATION

This application is a U.S. National Phase under 35 U.S.C. § 371 of International Application PCT/KR2020/018484 filed on Dec. 16, 2020, which are incorporated herein in their entirety. All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### SEQUENCE LISTING

The instant application contains a Sequence Listing which has been submitted electronically in XML format and is hereby incorporated by reference in its entirety. Said XML copy, created on Jun. 19, 2023, entitled Sequence Listing ST.26 (036794.000003), and is 32,000 bytes in size.

### FIELD OF THE INVENTION

The present invention relates to a nucleic acid construct that may express and inhibit a gene simultaneously.

### BACKGROUND OF THE INVENTION

Cancer is one of the biggest diseases that threatens the human health, and is a disease caused when cells proliferate and become immortalized in an unrestricted and uncontrolled manner through a series of mutation process. As various biochemical mechanisms related to cancer have been identified, therapeutic agents thereof have been developed, but a fundamental treatment method has not yet been proposed. Cancer immunotherapy is becoming a new paradigm for such cancer treatment.

The cancer immunotherapy may be divided into passive immunotherapy and active immunotherapy. In the passive immunotherapy, immune checkpoint inhibitors, immune cell therapy, therapeutic antibodies, and the like are used, and among them, the immune checkpoint inhibitor is a therapeutic agent that attacks cancer cells by activating T cells by blocking the activation of immune checkpoint proteins involved in suppressing T cells, and includes CTLA-4, PD-1, an PD-L1 inhibitor, and the like.

In the active immunotherapy, cancer treatment vaccines, immunomodulators, etc. are used, and specifically, the active immunotherapy is a cell therapy method that collects and reinforces immune cells in the body or transforms the immune cells through genetic engineering and introduces the transformed immune cells again, and includes tumor infiltrating lymphocyte (TIL), T cell receptor (TCR), chimeric antigen receptor (CAR) cell therapy products, and the like. There are cancer vaccines and the like that attack cancer cells by administering tumor-specific antigens of cancer cells to cancer patients to activate an immune system and activate an immune function in vivo.

Among them, dendritic cells (DC) are the most important antigen-presenting cells of the immune system, and dendritic cell cancer vaccines are considered as ideal therapeutic agents that may effectively remove tumors by presenting cancer antigens to activate anticancer immune responses. However, tumor and a tumor microenvironment (TME) directly induce dysfunction of dendritic cells or hide tumor antigens and secrete a large amount of immunosuppressive cytokines to inhibit anticancer immune activity. In particular, when the balance of power between cancer cells and immune cells to attack the cancer cells is broken, the cancer cells start to proliferate in earnest and disrupt an immune system in vivo, but at this time, some cancer cells avoid the immunity by using immune checkpoints of the immune cells.

Therefore, there is a need for a novel cancer immunotherapy that actively increases the immune response to cancer cells by activating the immune system and passively blocks the activation of immune checkpoint proteins so as not to interfere with immune escape.

In addition, it is necessary to smoothly up-regulate or down-regulate specific genes in a complex environment of a genome system, particularly in a cancer environment, but it is very difficult to achieve such gene regulation by treating one substance. That is, there have been many studies aimed at accurate and rapid regulation of genes in vivo using a single molecule, but sufficient results have not been obtained.

### SUMMARY OF THE INVENTION

The present inventors prepared a nucleic acid construct including mRNA and siRNA, and confirmed that the construct simultaneously expressed or inhibited specific genes through mRNA or siRNA, respectively. In particular, the present inventors confirmed that an immune escape mechanism by an immune checkpoint protein may be overcome by expressing antigen mRNA to activate the immune system and inhibit the activation of the immune checkpoint protein simultaneously, and then completed the present invention.

Accordingly, an object of the present invention is to provide a nucleic acid construct including mRNA and siRNA that may express and inhibit a gene simultaneously.

In order to achieve the object, the present invention provides a nucleic acid construct including mRNA and siRNA.

Further, the present invention provides a composition including the nucleic acid construct and a nucleic acid delivery system.

Further, the present invention provides a pharmaceutical composition for immunotherapy including the nucleic acid construct.

Further, the present invention provides a cancer vaccine including the nucleic acid construct.

Further, the present invention provides a method for preparing the nucleic acid construct.

According to the present invention, the nucleic acid construct that may express and inhibit a gene simultaneously includes both mRNA and siRNA in a single construct, and thus, mRNA and siRNA are introduced simultaneously into the same cell. Accordingly, a protein to be expressed may be expressed normally while expression of a particular gene may be specifically inhibited. The mRNA-siRNA nucleic acid construct activates an immune system by expressing a target protein in an antigen-presenting cell, and at the same time, overcomes interference of immune escape by blocking the activity of an immune checkpoint protein, and thus is expected to be remarkably useful in the fields of immunotherapy, especially in the field of cancer immunotherapy.

### BRIEF DESCRIPTION OF THE DRAWINGS

FIG. 1 illustrates a schematic diagram of a nucleic acid construct.

FIG. 2 is a schematic diagram sequentially illustrating a method for preparing an mRNA-siRNA nucleic acid construct according to the present invention.

FIG. 3 is a schematic diagram sequentially illustrating a method for preparing an mRNA-siRNA nucleic acid construct according to the present invention. A DNA template, mRNA (“mRNA-siRNA (SS or AS)”) transcribed in vitro using the template DNA, and an mRNA-siRNA nucleic acid construct prepared by annealing siRNA to the mRNA are illustrated together with indication of a sense sequence (SS) or antisense sequence (AS) of siRNA

FIG. 4 illustrates a process in which the mRNA-siRNA nucleic acid construct according to the present invention is separated into mRNA and siRNA by RNase H in an intracellular environment (letters of AAAA and TTTT correspond to DNA sequences).

FIG. 5 illustrates mRNA-siRNA examples prepared by annealing siRNA-Oligo (dT), in which a sense or antisense sequence of siRNA and Oligo (dT) of different lengths are combined in various configurations, to mRNA (letters of TT, TTTTTT, TTTT-AS, and TTTT-SS correspond to DNA sequences).

FIG. 6 is a diagram illustrating results of electrophoresis for a DNA template (a) and an in vitro transcription product (b).

FIG. 7 is a diagram illustrating results of electrophoresis for mRNA-siRNA.

FIG. 8 is a diagram illustrating results of evaluating protein (red fluorescent protein, GFP) expression ability of different mRNA-siRNA examples.

FIG. 9 is a diagram illustrating results of evaluating protein (blue fluorescent protein, BFP) expression ability and gene (green fluorescent protein, GFP) expression inhibition ability of different mRNA-siRNA examples after 24 hours of transfection. (a) mBFP protein expression efficiency, (b) GFP protein expression inhibition efficiency.

FIG. 10 is a diagram illustrating results of evaluating protein (blue fluorescent protein, BFP) expression ability and gene (green fluorescent protein, GFP) expression inhibition ability of different mRNA-siRNA examples after 48 hours of transfection. (a) mBFP protein expression efficiency, (b) GFP protein expression inhibition efficiency.

FIG. 11 is a diagram illustrating results of evaluating STAT3 protein expression inhibition ability of mOVA-siSTAT3 by introducing mRNA expressing ovalbumin (OVA) and siRNA inhibiting the expression of a STAT3 gene into the nucleic acid construct according to the present invention.

FIG. 12 is a diagram illustrating results of evaluating STAT3 protein expression inhibition ability of mOVA-siSTAT3 through western blot by introducing mRNA expressing ovalbumin (OVA) and siRNA inhibiting the expression of a STAT3 gene into the nucleic acid construct according to the present invention.

FIG. 13 is a diagram illustrating results of confirming an effect of mOVA-siSTAT3 on promoting maturation of dendritic cells through expression levels of CD40 and CD80.

FIG. 14 illustrates a result of confirming the expression of OVA through treatment of a mOVA-siSTAT3/siSTAT3 construct.

FIG. 15 illustrates results of analyzing an induced OVA antigen-specific cytotoxic T cell group as 13.8% among splenocytes collected from mice intramuscularly injected with mOVA-siSTAT3/siSTAT3.

FIG. 16 illustrates observation of a higher OVA-specific cytotoxic T cell group in splenocytes of mice intramuscularly injected with mOVA-siSTAT3/siSTAT3 than mice intramuscularly injected with a simple mixture of mOVA and siSTAT3.

FIG. 17 illustrates results of confirming that OVA-specific IgG expression in mice intramuscularly injected with mOVA-siSTAT3/siSTAT3 is improved compared to that of mice intramuscularly injected with a simple mixture of mOVA and siSTAT3.

FIG. 18 illustrates results of showing a significantly excellent tumor inhibition effect of siSTAT3/siSTAT3 (mOVA-T6-STAT3i) compared to a simple mixture.

### DETAILED DESCRIPTION OF THE INVENTION

Hereinafter, the present invention will be described in detail.

Unless defined otherwise, all technical and scientific terms used in the present specification have the same meaning as commonly understood by those skilled in the art to which the present invention pertains. In general, the nomenclature used herein is well-known and commonly used in the art.

As used herein, the term “vector” refers to a nucleic acid construct including essential regulatory elements operably linked to express a target protein in an appropriate host cell.

In the present invention, the vector may be used without limitation as long as it is suitable for producing the nucleic acid construct according to the present invention, and includes elements necessary for transcription such as a promoter. The vector may be an RNA vector or a DNA vector, preferably a DNA vector.

Any vector known to those skilled in the art, such as a viral vector or a plasmid vector, may be used as the vector without limitation, and preferably, a DNA plasmid vector may be used. Further, the vector may be characterized as a circular molecule. The vector, preferably the circular vector, may be linearized, for example by a restriction enzyme.

As used herein, the term “promoter” refers to a DNA sequence site to which transcriptional regulators bind, and for the purpose of the present invention, a promoter capable of inducing strong and stable gene expression may be used to increase a gene expression rate. In the present invention, the promoter may preferably be an RNA polymerase promoter, more preferably a T7 promoter or an SP6 promoter, but is not limited thereto.

As used herein, the term “untranslation region (UTR)” refers to an untranslation region of mRNA and generally means both ends of a coding region. In particular, a 5′ end part is referred to as a 5′ UTR, and a 3′ end part is referred to as a 3′ UTR.

As used herein, the term “poly (A) tail” is also called polyadenylic acid or polyadenylic acid fragment, and means a sequence of continuous adenylic acids commonly present at the 3′ end of eukaryotic mRNA. The length is about 10 to 200 nucleotides (nt), and may be variously adjusted according to an allowable size of an expression vector backbone. The poly (A) is known to be involved in stabilization, translation, transport from the nucleus to the cytoplasm, and the like of mRNA.

As used herein, the term “small interfering RNA (siRNA)” is a small RNA fragment of 18 to 23 nucleotides (nt) in size produced by cleavage of double-stranded RNA by a Dicer enzyme, and may be used by specifically binding to mRNA having a complementary sequence to inhibit the expression of a target protein, mRNA. In the siRNA, sense RNA and antisense RNA form a double-stranded RNA molecule, and at this time, the sense RNA is preferably a siRNA molecule including the same nucleic acid sequence as a target sequence of some contiguous nucleotides of mRNA. The antisense sequence has most preferably a complementary sequence to the sense sequence.

As used herein, the term “complementary sequence” may include 60 to 100% of a complementary sequence, preferably 80% to 100% of a complementary sequence, more preferably 90% to 100% of a complementary sequence, much more preferably 95% to 100% of a complementary sequence in addition to 100% of a complementary sequence, as long as it may maintain a characteristics of forming complementary binding to each other.

As used herein, the term “target gene” is a gene of which the expression is

selectively inhibited or inactivated by the siRNA. This inactivation is achieved when the siRNA cleaves mRNA of the target gene.

As used herein, the term “ribonuclease (RNase)” is an enzyme that catalyzes a reaction to decompose ribonucleic acid into oligonucleotides or single nucleic acids, preferably endo ribonuclease, and the endo ribonuclease includes RNase A, RNase H, RNase I, RNase III, RNase L, RNase P, RNase PhyM, RNase T1, RNase T2, RNase U2, RNase V1 and RNase V.

In the present invention, the ribonucleic acid may be messenger RNA, and RNase H may be preferably used as the ribonuclease. In one example of the present invention, it is confirmed that RNase H is used to hydrolyze a phosphodiester bond of a poly (A)-poly (T) hybridized portion of mRNA-siRNA, which is the nucleic acid construct according to the present invention, so that it is possible to achieve gene expression according to mRNA and inhibition of the protein expression according to siRNA simultaneously.

As used herein, the term “mRNA-siRNA (SS or AS)” refers to mRNA including a sense or antisense sequence of siRNA at a 3′ end. In one example of the present invention, a poly (T) tail and an siRNA (antisense/sense) sequence are introduced into 3′ next to 3′UTR and siRNA (antisense/sense) using a reverse primer (RP) including a poly (A) sequence and/or a siRNA (sense/antisense) sequence to prepare a DNA template, and mRNA (“mRNA-siRNA (SS or AS)”) including an siRNA sense or antisense sequence at the 3′ end was prepared through in vitro transcription (IVT) using the prepared DNA template.

As used herein, the term “mRNA-siRNA” refers to a nucleic acid construct in which a poly (A) tail and a siRNA sequence (sense/antisense) at the 3′ end of mRNA-siRNA bind to a hybridization sequence (antisense/sense) and oligo (dT) complementary thereto, respectively, to form a double strand, as a result of annealing the “hybridization sequence” capable of complementarily binding to the sense or antisense sequence of siRNA introduced to the 3′ end of the prepared mRNA-siRNA (SS or AS).

In the present invention, the “hybridization sequence” may be siRNA or DNA, and thus, “mRNA-siRNA” is also referred to as “mRNA-siRNA/siRNA” or “mRNA-siRNA/DNA” in the present specification.

The present invention provides a nucleic acid construct that may express and inhibit a gene simultaneously.

More specifically, the present invention provides a nucleic acid construct including mRNA and siRNA, the nucleic acid construct including (i) mRNA including a poly (A) tail; and a first siRNA sense or antisense sequence sequentially located from a 5′ direction to a 3′ direction at a 3′ end; and (ii) a hybridization sequence that complementarily binds to the first siRNA sequence.

In the present invention, the hybridization sequence is a second siRNA antisense or sense sequence complementarily binding to the first siRNA sequence; or a DNA antisense or sense sequence complementarily binding to the first siRNA sequence.

In the present invention, the hybridization sequence may further include a poly (T) tail complementarily binding to the poly (A) tail of the mRNA at the 3′ end. The poly (T) tail may have a length of 2 to 6 nt, but is not limited thereto.

In the present invention, the nucleic acid construct may further include a first linker sequence for an additional siRNA sense or antisense sequence at the 3′ end of the first siRNA sense or antisense sequence, and the first linker sequence may further include an additional siRNA sense or antisense sequence at the 3′ end of the linker sequence.

In addition, in the present invention, the nucleic acid construct may further include a hybridization sequence complementarily binding to the first linker sequence; and the additional siRNA sense or antisense sequence; and a second linker sequence. The hybridization sequence complementarily binds to the first linker sequence; and the additional siRNA sense or antisense sequence.

In the present invention, as a linker or adapter sequence, synthetic oligonucleotide sequences according to a conventional method may be used without limitation, and preferably, the first linker sequence may be a poly (U) tail, and the second linker sequence may be a poly (A) tail complementarily binding to the first linker sequence, but are not limited thereto.

The linker or adapter sequence may include a region that does not hybridize with DNA.

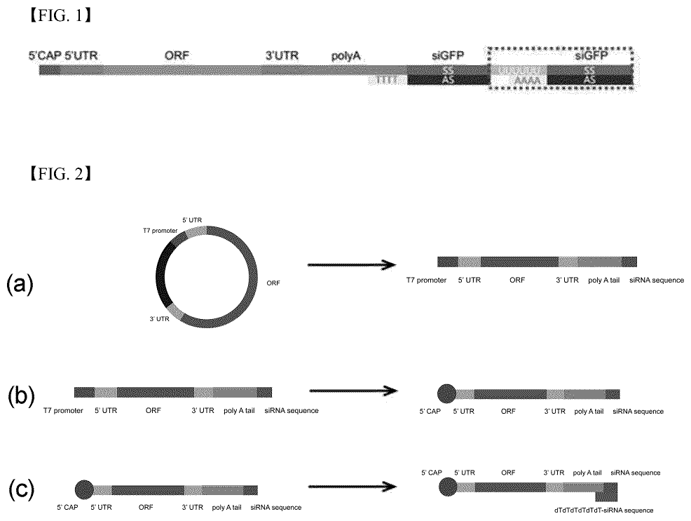

In the present invention, the nucleic acid construct may further include a plurality of repeating units indicated by red dotted lines in FIG. 1 below, and preferably 1 to 5 repeating units. The number of repeating units to be added may be appropriately adjusted by those skilled in the art according to the purpose.

Specifically, the nucleic acid construct may further include a plurality of repeating units including (a) and (b) at the 3′ end of the first siRNA sense or antisense sequence, and the (a) and (b) include (a) a first linker sequence for an additional siRNA sense or antisense sequence; and an additional siRNA sense or antisense sequence; and (b) a hybridization sequence; and a second linker sequence, and at this time, the hybridization sequence complementarily binds to the first linker sequence; and the additional siRNA sense or antisense sequence.

siRNAs to be added as part of repeating units may have the same or different target genes, and when the nucleic acid construct is injected into a cell, the hybridization site of mRNA-siRNA is cleaved by RNase H and separated into independent double-stranded siRNA, so as to effectively inhibit the expression of the target gene. When two or more different siRNAs are included, the expression of two or more target genes may be inhibited simultaneously.

Further, the present invention provides a composition including the nucleic acid construct according to the present invention and a nucleic acid delivery system.

As used herein, the term “nucleic acid delivery system” is intended to increase the delivery efficiency of the nucleic acid construct according to the present invention into the body, and has an advantage of having excellent stability in the body and a simple manufacturing process as a drug.

The nucleic acid delivery system may include, but is not limited thereto, viral vectors, non-viral vectors, liposomes, cationic polymers, micelles, emulsions, and nanoparticles, preferably nanoparticles. The cationic polymers include natural polymers such as chitosan, atelocollagen, and cationic polypeptide, and synthetic polymers such as poly (L-lysin), linear or branched polyethylene imine (PEI), cyclodextrin-based polycations and dendrimer.

In the present invention, the nanoparticles, may be, but are not limited thereto, any one selected from the group consisting of magnetic beads, gold (Au) nanoparticles, silver (Ag) nanoparticles, platinum (Pt) nanoparticles, quantum dots, upconversion nanoparticles (UCNP), graphene-nanoparticle composites, color dyed particles and latex nanoparticles, silica nanoparticles, carbon derivative nanoparticles; e.g., carbon nanotubes and solid lipid nanoparticles.

When the nucleic acid construct according to the present invention is introduced into the nanoparticles, the delivery of the nucleic acid construct to the target cell is promoted, and the nucleic acid construct is delivered to the target cell even at a relatively low dose, thereby exhibiting high target protein expression induction and target gene expression inhibition functions. In addition, it is possible to prevent delivery of non-specific mRNA-siRNA to other organs and cells other than the target.

Further, the present invention provides a pharmaceutical composition for immunotherapy including the nucleic acid construct according to the present invention. The pharmaceutical composition according to the present invention may increase an immune response or selectively increase some of immune responses preferable for treatment or prevention of a specific disease, infection or disorder.

In the present invention, the immunotherapy may be preferably cancer immunotherapy.

In the present invention, the cancer may be any one selected from the group consisting of brain tumor, benign astrocytoma, malignant astrocytoma, pituitary adenoma, meningioma, brain lymphoma, oligodendroglioma, craniopharyngioma, ependymoma, brainstem tumor, head and neck tumor, laryngeal cancer, oropharyngeal cancer, nasal/sinus cancer, nasopharyngeal cancer, salivary gland cancer, hypopharyngeal cancer, thyroid cancer, chest tumor, small cell lung cancer, non-small cell lung cancer, thymus cancer, mediastinal tumor, esophageal cancer, breast cancer, male breast cancer, abdominal tumor, stomach cancer, liver cancer, gallbladder cancer, biliary tract cancer, pancreatic cancer, small intestine cancer, colorectal cancer, anal cancer, bladder cancer, kidney cancer, male genital tumor, penile cancer, urethral cancer, prostate cancer, female genital tumor, cervical cancer, endometrial cancer, ovarian cancer, uterine sarcoma, vaginal cancer, female external genital cancer, female urethral cancer, skin cancer, myeloma, leukemia and malignant lymphoma, but is not limited thereto.

In the present invention, the pharmaceutical composition may be provided by including the nucleic acid construct alone according to the present invention or by including one or more pharmaceutically acceptable carriers, excipients or diluents.

The “pharmaceutically acceptable” refers to a composition that is physiologically acceptable and does not cause an allergic reaction, such as gastrointestinal disorder, dizziness, etc., or similar reactions thereto when administered to humans. Examples of the carrier, the excipient, and the diluent may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil, but are not limited thereto.

The pharmaceutical composition according to the present invention may further include a lubricant, a wetting agent, a sweetening agent, a flavoring agent, an emulsifying agent, a suspending agent, a preservative, and the like, in addition to the ingredients. Solid formulations for oral administration include tablets, pills, powders, granules, capsules, and the like, and these solid formulations may be prepared by mixing at least one or more excipients, for example, starch, calcium carbonate, sucrose or lactose, gelatin, and the like to the ceftezole. Further, lubricants such as magnesium stearate and talc are used in addition to simple excipients. Liquid formulations for oral administration may correspond to suspensions, oral liquids, emulsions, syrups, and the like, and may include various excipients, for example, a wetting agent, a sweetener, an aromatic agent, a preservative, and the like, in addition to water and liquid paraffin which are commonly used as simple diluents. Formulations for parenteral administration include sterilized aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

The preferable dose of the pharmaceutical composition varies according to the condition and body weight of a patient, the severity of a disease, a drug form, and route and period of administration, but may be properly selected by those skilled in the art. However, for preferable effects, the pharmaceutical composition may be administered in an amount of 0.0001 to 100 mg/kg per day, preferably 0.001 to 100 mg/kg. The administration may be performed once a day or several times a day. The dose does not limit the scope of the present invention in any aspect.

The pharmaceutical composition may be administered by various routes, for example, oral, rectal or intravenous, intramuscular, subcutaneous, intraperitoneal, intrauterine intrathecal or intracerebrovascular injection.

Further, the present invention provides a cancer vaccine including the nucleic acid construct according to the present invention. The nucleic acid construct according to the present invention may induce an immune response by labeling an antigen on an antigen-presenting cell, and simultaneously inhibit the expression of an immune checkpoint protein. The nucleic acid construct may be included in a cancer vaccine in a state bound to the nanoparticles.

In the present invention, the cancer vaccine may further include dendritic cells. For example, the cancer vaccine may be provided in the form of delivering the nucleic acid construct according to the present invention to dendritic cells ex vivo and then delivering the transfected dendritic cells. The dendritic cells may increase the immunogenicity of a subject, thereby effectively preventing or inhibiting tumor proliferation and/or metastasis in the subject.

As used herein, the term “dendritic cell” refers to an antigen presenting cell that absorbs an antigen into the cell, treats the antigen, and presents an antigen or a peptide derived from the antigen together with an MHC class I complex or an MHC class complex. The dendritic cells are classified into immature dendritic cells and mature dendritic cells according to maturity.

The “immature dendritic cells” are found in the early stage of maturation, and refer to dendritic cells that do not express cell surface markers such as CD14, like mature dendritic cells, express low levels of HLA-DR, CD86, CD80, CD83 or CD40 and express normal levels of CD1a and CCR1, CCR2, CCR5 and CXCR1.

The “mature dendritic cells” refer to cells formed by maturation of immature dendritic cells, and refer to cells that express cell surface markers involved in the activities of B cells and T cells, such as MHC class I or MHC class HLA-DR, cell adhesion factors CD54, CD18, CD11, costimulatory factors (e.g., CD86, CD80, CD83 or CD40) at higher or relatively increased levels than immature dendritic cells.

For example, in one example of the present invention, as a result of transfecting the nucleic acid construct according to the present invention with mRNA-siSTAT3 introduced with mRNA expressing ovalbumin (OVA) and siRNA inhibiting the expression of the STAT3 gene, it was confirmed that the expression levels of the CD40 and CD80 markers were remarkably increased, and thus the effect of promoting dendritic cell maturation was much better than that of mixing and administering the separated mRNA and siRNA.

The present invention also provides a pharmaceutical composition including the nucleic acid construct according to the present invention for use in immunotherapy.

The present invention also provides a use of the nucleic acid construct according to the present invention in the preparation of a drug for immunotherapy.

The present invention also provides an immunotherapy method including administering the nucleic acid construct according to the present invention to a subject in need thereof.

In the present invention, the immunotherapy may be cancer immunotherapy. More specifically, the immunotherapy may be used for cancer treatment.

Further, the present invention provides a method for preparing a nucleic acid construct including mRNA and siRNA, including:
1) preparing a DNA template into which a poly (T) tail and a siRNA antisense or sense sequence located sequentially from a 5′ direction to a 3′ direction at a 3′ end are introduced;
2) obtaining mRNA including the siRNA sequence at the 3′ end by treating the DNA template with a reaction solution containing RNA polymerase; and
3) annealing siRNA having oligo (dT) linked at the 3′ end to the mRNA product.

In step 1), the DNA template is a transcriptional template for mRNA synthesis, and a DNA vector (pUCIDT-Amp vector) including a T7 promoter, a 5′ UTR, an open reading frame (ORF) and a 3′ UTR was prepared, and PCR was performed using the DNA as a template to linearize and then amplify a vector. The PCR was performed by using a PCR reaction mixture using the DNA

vector as a primary template, an oligonucleotide consisting of 5′-TTGGACCCTCGTACAGAAGCTAATACG-3′ (SEQ ID NO: 4) as a forward primer, and a configuration of “5′-siRNA sequence-poly (A)-primer sequence specifically binding to DNA vector-3′ (reverse primer)” as a reverse primer. Specifically, the reverse primer includes a poly (A) sequence and/or a siRNA (sense/antisense) sequence to introduce a poly (T) tail and a siRNA (antisense/sense) sequence to 3′ next to the 3′ UTR of the prepared DNA template. In one example of the present invention, 2× TOPsimple™ DryMIX-HOT (enzynomics, Korea) was used as the PCR reaction mixture, but is not limited thereto.

The PCR conditions for preparing the DNA template may maintain the temperatures and times for performing three steps of PCR, that is, a denaturation step, an annealing step, and an elongation step, respectively. The conditions for performing the denaturation step may be 85° C. to 105° C. for 5 minutes to 15 minutes, preferably 95° C. for 10 minutes, the conditions for performing the hybridization step may be 40° C. to 65° C. for 20 seconds to 40 seconds, preferably 59° C. for 30 seconds, and the conditions for performing the extension step may be 50° C. to 80° C. for 30 seconds to 2 minutes, preferably 72° C. for 1 minute. The specified temperature and time range for performing the PCR may be adjusted within a feasible range in performing the PCR.

In one example of the present invention, the PCR conditions for preparing the DNA template were performed by total 30 cycles of denaturation at 95° C. and 3 seconds, hybridization at 59° C. and 30 seconds, and extension at 72° C. and 1 minute 1 after initial denaturation at 95° C. and 10 minutes in one cycle, and final elongation at 72° C. and 5 minutes. In addition, the amplified PCR reaction product was purified using AccuPrep®PCR Purification Kit (Bioneer, South Korea).

Step 2) is an in vitro transcription (IVT) step, and may be characterized by preparing mRNA (“mRNA-siRNA”) including a siRNA sequence at the 3′ end by treating the DNA template prepared in step 1) with the reaction solution containing RNA polymerase.

In one example of the present invention, the in vitro transcription was performed by treating the prepared DNA template with the reaction solution, culturing at 37° C. for 16 hours, adding 1 μl of Turbo DNase I, and reacting at 37° C. for 15 minutes. However, specific reaction conditions for the in vitro transcription may be controlled within a feasible range.

In step 3), the conditions for annealing are sequence-dependent and vary depending on environmental variables, but may be 40 to 80° C. for 5 minutes to 15 minutes, preferably 75° C. for 10 minutes. In one example of the present invention, the annealing was performed by fast cooling (quick cooling) to 4° C. after performed at 75°° C. for 10 minutes.

Hereinafter, the present invention will be described in detail by Examples. However, the following Examples are merely illustrative of the present invention, and those skilled in the art will understand that various modifications and other equivalent Examples are possible therefrom. Accordingly, the true technical scope of the present invention should be defined by the technical spirit of the appended claims.

### Example. Preparation of Nucleic Acid Construct According to the Present Invention

### Preparation of DNA Template

In order to prepare a nucleic acid construct capable of simultaneously expressing and suppressing a gene, an experiment was performed as follows. First, a DNA vector (pUCIDT-Amp vector) including a T7 promoter, a 5′UTR, an open reading frame (ORF), and a 3′UTR was prepared as a transcription template for mRNA synthesis. The vector was linearized and amplified from the T7 promoter to the 3′UTR by performing PCR using the DNA as a template. PCR was performed using TOPsimple™ DryMIX-HOT (enzynomics, Korea), and specific PCR conditions were shown in Table 1 below. The amplified PCR reaction product was purified using AccuPrep®PCR Purification Kit (Bioneer, South Korea).

TABLE 1
Standard reaction conditions

| PCR mixture | |
|---|---|
| 2X TOPsimple ™ DryMIX-HOT | 1 tube |
| Template DNA (3-5 ng/μl) | 1 μl |
| Forward Primer (10 pmole/μl) | 1 μl |
| Reverse Primer (10 pmole/μl) | 1 μl |
| DEPC water | up to 20 μl |

PCR cycles

| Step | Temperature | Time | | Cycle |
|---|---|---|---|---|
| Initial denaturation* | 95° C. | 10 | min | 1 |
| Denaturation | 95° C. | 30 | sec | 30 |
| Annealing* | 59° C. | 30 | sec | |
| Elongation | 72° C. | 1 | min/kb | |
| Final elongation | 72° C. | 5 | min | 1 |

At this time, using a reverse primer (RP) including a poly (A) sequence and/or an siRNA (sense/antisense) sequence, a DNA template was prepared in which a poly (T) tail and an siRNA (antisense/sense) sequence were introduced to 3′ next to the 3′UTR (FIG. 2A). That is, the reverse primer was used based on the following configuration.

5′—siRNA sequence—poly (A)—primer sequence specifically binding to DNA vector—3′ (reverse primer)

In Example, the present inventors used a primer consisting of only a 120 nt poly (A) sequence as the reverse primer, a primer including a 120 nt poly (A) sequence and a siRNA (sense/antisense) sequence, siRNA (hereinafter, siGFP) targeting green fluorescent protein (GFP) mRNA, and mRNA encoding A red fluorescent protein (RFP) and A blue fluorescent protein (BFP).

### mRNA-siRNA Synthesis

In vitro transcription (IVT) was performed using the DNA template prepared in Example to synthesize mRNA (hereinafter referred to as “mRNA-siRNA (SS or AS)”) including an siRNA sequences at the end. The in vitro transcription was performed using a reaction solution (MEGAscript™ T7 Transcription Kit (Ambion, USA) including T7 RNA polymerase, rATP, rCTP, IGTP, rUTP and a cap analogue, and the template DNA was removed by treatment of DNase and only the mRNA was recovered.

Specifically, the reaction solution was put into a tube and incubated at 37° C. for 16 hours, then added with 1 μl of Turbo DNase I, and reacted at 37° C. for 15 minutes, and a reaction product was purified using MEGAclear™ Kit (Ambion, USA). All components constituting the reaction solution and capacities and concentrations thereof were shown in Table 2 below. In the mRNA synthesized through this, a 5′-cap produced by adding the cap analog to the transcription reaction was included at the 5′ end (FIG. 2B).

TABLE 2

| Amount | Component | Concentration |
|---|---|---|
| to 20 μl | DEPC water | |

| | | | | |
|---|---|---|---|---|
| 5 | μl | 3′-O-Me-m7G(5′)ppp(5′)G RNA Cap | 10 | mM |
| | | Structure Analog (NEB, USA) | | |
| 2 | μl | ATP solution | 75 | mM |
| 1.5 | μl | mCTP solution(Trilink, USA) | 100 | mM |
| 0.5 | μl | GTP solution | 75 | mM |
| 1.5 | μl | Pseudo UTP solution (Trilink, USA) | 100 | mM |
| 1 | μg | PCR amplified DNA template | | |

| | | | |
|---|---|---|---|
| 2 | μl | Enzyme mix | 10X |
| 2 | μl | 10x reaction buffer | 10X |

### Preparation of Nucleic Acid Construct According to the Present Invention

Next, a reverse primer including siRNA (siRNA-Oligo (dT)) linked to dTdTdTdTdTdT was annealed to the mRNA-siRNA (SS or AS) prepared in Example above, a portion of the mRNA-siRNA (SS or AS) complementarily bound to a hybridization sequence to form a double-strand and then a nucleic acid construct (“mRNA-siRNA”) according to the present invention was prepared. In this case, the hybridization sequence corresponded to a poly (T)-containing siRNA sequence or a poly (T)-containing DNA sequence.

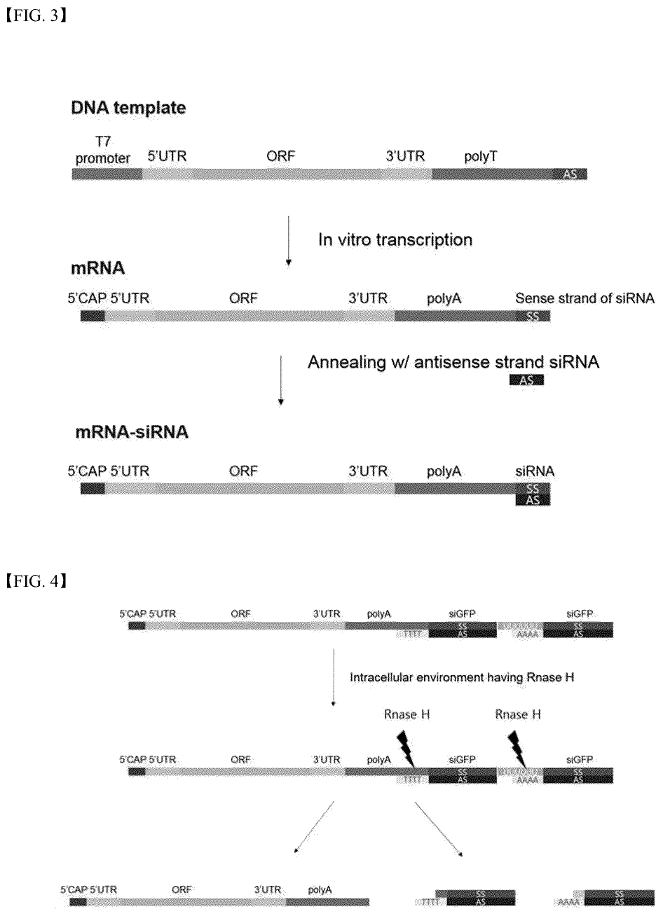

Specifically, for annealing, the mRNA-siRNA (SS or AS) and the siRNA-Oligo (dT) were put and mixed in the same tube, reacted at 75° C. for 10 minutes, and then fast-cooled (rapidly cooled) to 4° C. At this time, the dTdTdTdTdTdT site was annealed to the poly (A) tail of the mRNA-siRNA (SS or AS), the siRNA sequence (antisense/sense) was annealed complementarily to the siRNA introduced at the 3′ end of the mRNA-siRNA (SS or AS) (FIG. 2C), and as a result, the poly (A) tail and the siRNA sequence (sense/antisense) at the 3′ end of the mRNA-siRNA (SS or AS) bound to siRNA (antisense/sense) and oligo (dT) complementary thereto, respectively, to form a double strand and then the nucleic acid construct according to the present invention was prepared (hereinafter referred to as “(or mRNA-siRNA/siRNA or mRNA-siRNA/DNA)”). The preparing process of the mRNA-siRNA was schematically shown in FIG. 3.

The DNA vectors, all the primer sequences, and all the siRNA sequences used in Example and Experimental Examples were shown in Table 3 below. All the sequences were provided and used from IDT (Gene Synthesis Service).

TABLE 3

| | | SEQ ID |
|---|---|---|
| Gene name | Sequence information | NO: |
| pUCIDT- | TCGCGCGTTTCGGTGATGACGGTGAAAACCTCTG | 1 |
| AMP_mTagBF | ACACATGCAGCTCCCGGAGACGGTCACAGCTTGT | |
| P2 | CTGTAAGCGGATGCCGGGAGCAGACAAGCCCGT | |
| | CAGGGCGCGTCAGCGGGTGTTGGCGGGTGTCGG | |
| | GGCTGGCTTAACTATGCGGCATCAGAGCAGATTG | |
| | TACTGAGAGTGCACCAAATGCGGTGTGAAATAC | |
| | CGCACAGATGCGTAAGGAGAAAATACCGCATCA | |
| | GGCGCCATTCGCCATTCAGGCTGCGCAACTGTTG | |
| | GGAAGGGCGATCGGTGCGGGCCTCATCGCTATT | |
| | ACGCCAGCTGGCGAAAGGGGGATGTGCTGCAAG | |
| | GCGATTAAGTTGGGTAACGCCAGGGTTTTCCCAG | |
| | TCACGACGTTGTAAAACGACGGCCAGTGCAACG | |
| | CGATGACGATGGATAGCGATTCATCGATGAGCT | |
| | GACCCGATCGCCGCCGCCGGAGGGTTGCGTTTGA | |
| | GACGGGCGACAGATGAGGCTCGTTTAGTGAACC | |
| | GTCAGATCGCCTGGAGACGCCATCCACGCTGTTT | |
| | TGACCTCCATAGAAGACACCGGGACCGATCCAG | |
| | CCTCCGGACTCTAGAGGATCGAACCCTTTTGGAC | |
| | CCTCGTACAGAAGCTAATACGACTCACTATAGGG | |
| | AAATAAGAGAGAAAAGAAGAGTAAGAAGAAAT | |
| | ATAAGAGCCACCATGGTGTCTAAGGGCGAAGAG | |
| | CTGATTAAGGAGAACATGCACATGAAGCTGTAC | |
| | ATGGAGGGCACCGTGGACAACCATCACTTCAAG | |
| | TGCACATCCGAGGGCGAAGGCAAGCCCTACGAG | |
| | GGCACCCAGACCATGAGAATCAAGGTGGTCGAG | |
| | GGCGGCCCTCTCCCCTTCGCCTTCGACATCCTGG | |
| | CTACTAGCTTCCTCTACGGCAGCAAGACCTTCAT | |
| | CAACCACACCCAGGGCATCCCCGACTTCTTCAAG | |
| | CAGTCCTTCCCTGAGGGCTTCACATGGGAGAGAG | |
| | TCACCACATACGAAGACGGGGGCGTGCTGACCG | |
| | CTACCCAGGACACCAGCCTCCAGGACGGCTGCCT | |
| | CATCTACAACGTCAAGATCAGAGGGGTGAACTT | |
| | CACATCCAACGGCCCTGTGATGCAGAAGAAAAC | |
| | ACTCGGCTGGGAGGCCTTCACCGAGACGCTGTAC | |
| | CCCGCTGACGGCGGCCTGGAAGGCAGAAACGAC | |
| | ATGGCCCTGAAGCTCGTGGGCGGGAGCCATCTG | |
| | ATCGCAAACGCCAAGACCACATATAGATCCAAG | |
| | AAACCCGCTAAGAACCTCAAGATGCCTGGCGTCT | |
| | ACTATGTGGACTACAGACTGGAAAGAATCAAGG | |
| | AGGCCAACAACGAGACCTACGTCGAGCAGCACG | |
| | AGGTGGCAGTGGCCAGATACTGCGACCTCCCTA | |
| | GCAAACTGGGGCACAAGCTTAATGCTGCCTTCTG | |
| | CGGGGCTTGCCTTCTGGCCATGCCCTTCTTCTCTC | |
| | CCTTGCACCTGTACCTCTTGGTCTTTGAATAAAG | |
| | CCTGAGTAGGAAGTGAGGGTCTAGAACTAGTGT | |
| | CGACGCAAATCAGTTCTGGACCAGCGAGCTGTG | |
| | CTGCGACTCGTGGCGTAATCATGGTCATAGCTGT | |
| | TTCCTGTGTGAAATTGTTATCCGCTCACAATTCC | |
| | ACACAACATACGAGCCGGAAGCATAAAGTGTAA | |
| | AGCCTGGGGTGCCTAATGAGTGAGCTAACTCAC | |
| | ATTAATTGCGTTGCGCTCACTGCCCGCTTTCCAG | |
| | TCGGGAAACCTGTCGTGCCAGCTGCATTAATGAA | |
| | TCGGCCAACGCGCGGGGAGAGGCGGTTTGCGTA | |
| | TTGGGCGCTCTTCCGCTTCCTCGCTCACTGACTCG | |
| | CTGCGCTCGGTCGTTCGGCTGCGGCGAGCGGTAT | |
| | CAGCTCACTCAAAGGCGGTAATACGGTTATCCAC | |
| | AGAATCAGGGGATAACGCAGGAAAGAACATGTG | |
| | AGCAAAAGGCCAGCAAAAGGCCAGGAACCGTAA | |
| | AAAGGCCGCGTTGCTGGCGTTTTTCCATAGGCTC | |
| | CGCCCCCCTGACGAGCATCACAAAAATCGACGC | |
| | TCAAGTCAGAGGTGGCGAAACCCGACAGGACTA | |
| | TAAAGATACCAGGCGTTTCCCCCTGGAAGCTCCC | |
| | TCGTGCGCTCTCCTGTTCCGACCCTGTCGCTTACC | |
| | GGATACCTGTCCGCCTTTCTCCCTTCGGGAAGCG | |
| | TGGCGCTTTCTCATAGCTCACGCTGTAGGTATCT | |
| | CAGTTCGGTGTAGGTCGTTCGCTCCAAGCTGGGC | |
| | TGTGTGCACGAACCCCCCGTTCAGCCCGACCGCT | |
| | GCGCCTTATCCGGTAACTATCGTCTTGAGTCCAA | |
| | CCCGGTAAGACACGACTTATCGCCACTGGCAGC | |
| | AGCCACTGGTAACAGGATTAGCAGAGCGAGGTA | |
| | TGTAGGCGGTGCTACAGAGTTCTTGAAGTGGTGG | |
| | CCTAACTACGGCTACACTAGAAGAACAGTATTTG | |
| | GTATCTGCGCTCTGCTGAAGCCAGTTACCTTCGG | |
| | AAAAAGAGTTGGTAGCTCTTGATCCGGCAAACA | |
| | AACCACCGCTGGTAGCGGTGGTTTTTTTGTTTGC | |
| | AAGCAGCAGATTACGCGCAGAAAAAAAGGATCT | |
| | CAAGAAGATCCTTTGATCTTTTCTACGGGGTCTG | |
| | ACGCTCAGTGGAACGAAAACTCACGTTAAGGGA | |
| | TTTTGGTCATGAGATTATCAAAAAGGATCTTCAC | |
| | CTAGATCCTTTTAAATTAAAAATGAAGTTTTAAA | |
| | TCAATCTAAAGTATATATGAGTAAACTTGGTCTG | |
| | ACAGTTACCAATGCTTAATCAGTGAGGCACCTAT | |
| | CTCAGCGATCTGTCTATTTCGTTCATCCATAGTTG | |
| | CCTGACTCCCCGTCGTGTAGATAACTACGATACG | |
| | GGAGGGCTTACCATCTGGCCCCAGTGCTGCAATG | |
| | ATACCGCGAGACCCACGCTCACCGGCTCCAGATT | |
| | TATCAGCAATAAACCAGCCAGCCGGAAGGGCCG | |
| | AGCGCAGAAGTGGTCCTGCAACTTTATCCGCCTC | |
| | CATCCAGTCTATTAATTGTTGCCGGGAAGCTAGA | |
| | GTAAGTAGTTCGCCAGTTAATAGTTTGCGCAACG | |
| | TTGTTGCCATTGCTACAGGCATCGTGGTGTCACG | |
| | CTCGTCGTTTGGTATGGCTTCATTCAGCTCCGGTT | |
| | CCCAACGATCAAGGCGAGTTACATGATCCCCCAT | |
| | GTTGTGCAAAAAAGCGGTTAGCTCCTTCGGTCCT | |
| | CCGATCGTTGTCAGAAGTAAGTTGGCCGCAGTGT | |
| | TATCACTCATGGTTATGGCAGCACTGCATAATTC | |
| | TCTTACTGTCATGCCATCCGTAAGATGCTTTTCTG | |
| | TGACTGGTGAGTACTCAACCAAGTCATTCTGAGA | |
| | ATAGTGTATGCGGCGACCGAGTTGCTCTTGCCCG | |
| | GCGTCAATACGGGATAATACCGCGCCACATAGC | |
| | AGAACTTTAAAAGTGCTCATCATTGGAAAACGTT | |
| | CTTCGGGGCGAAAACTCTCAAGGATCTTACCGCT | |
| | GTTGAGATCCAGTTCGATGTAACCCACTCGTGCA | |
| | CCCAACTGATCTTCAGCATCTTTTACTTTCACCAG | |
| | CGTTTCTGGGTGAGCAAAAACAGGAAGGCAAAA | |
| | TGCCGCAAAAAAGGGAATAAGGGCGACACGGAA | |
| | ATGTTGAATACTCATACTCTACCTTTTTCAATATT | |
| | ATTGAAGCATTTATCAGGGTTATTGTCTCATGAG | |
| | CGGATACATATTTGAATGTATTTAGAAAAATAAA | |
| | CAAATAGGGGTTCCGCGCACATTTCCCCGAAAA | |
| | GTGCCACCTGACGTCTAAGAAACCATTATTATCA | |
| | TGACATTAACCTATAAAAATAGGCGTATCACGA | |
| | GGCCCTTTCGTC | |
| pUCIDT- | TCGCGCGTTTCGGTGATGACGGTGAAAACCTCTG | 2 |
| AMP_DsRed | ACACATGCAGCTCCCGGAGACGGTCACAGCTTGT | |
| Express2 RFP | CTGTAAGCGGATGCCGGGAGCAGACAAGCCCGT | |
| | CAGGGCGCGTCAGCGGGTGTTGGCGGGTGTCGG | |
| | GGCTGGCTTAACTATGCGGCATCAGAGCAGATTG | |
| | TACTGAGAGTGCACCAAATGCGGTGTGAAATAC | |
| | CGCACAGATGCGTAAGGAGAAAATACCGCATCA | |
| | GGCGCCATTCGCCATTCAGGCTGCGCAACTGTTG | |
| | GGAAGGGCGATCGGTGCGGGCCTCATCGCTATT | |
| | ACGCCAGCTGGCGAAAGGGGGATGTGCTGCAAG | |
| | GCGATTAAGTTGGGTAACGCCAGGGTTTTCCCAG | |
| | TCACGACGTTGTAAAACGACGGCCAGTGCAACG | |
| | CGATGACGATGGATAGCGATTCATCGATGAGCT | |
| | GACCCGATCGCCGCCGCCGGAGGGTTGCGTTTGA | |
| | GACGGGCGACAGATGAGGCTCGTTTAGTGAACC | |
| | GTCAGATCGCCTGGAGACGCCATCCACGCTGTTT | |
| | TGACCTCCATAGAAGACACCGGGACCGATCCAG | |
| | CCTCCGGACTCTAGAGGATCGAACCCTTTTGGAC | |
| | CCTCGTACAGAAGCTAATACGACTCACTATAGGG | |
| | AAATAAGAGAGAAAAGAAGAGTAAGAAGAAAT | |
| | ATAAGAGCCACCATGGATAGCACTGAGAACGTC | |
| | ATCAAGCCCTTCATGCGCTTCAAGGTGCACATGG | |
| | AGGGCTCCGTGAACGGCCACGAGTTCGAGATCG | |
| | AGGGCGAGGGCGAGGGCAAGCCCTACGAGGGCA | |
| | CCCAGACCGCCAAGCTGCAGGTGACCAAGGGCG | |
| | GCCCCCTGCCCTTCGCCTGGGACATCCTGTCCCC | |
| | CCAGTTCCAGTACGGCTCCAAGGTGTACGTGAAG | |
| | CACCCCGCCGACATCCCCGACTACAAGAAGCTGT | |
| | CCTTCCCCGAGGGCTTCAAGTGGGAGCGCGTGAT | |
| | GAACTTCGAGGACGGCGGCGTGGTGACCGTGAC | |
| | CCAGGACTCCTCCCTGCAGGACGGCACCTTCATC | |
| | TACCACGTGAAGTTCATCGGCGTGAACTTCCCCT | |
| | CCGACGGCCCCGTAATGCAGAAGAAGACTCTGG | |
| | GCTGGGAGCCCTCCACCGAGCGCCTGTACCCCCG | |
| | CGACGGCGTGCTGAAGGGCGAGATCCACAAGGC | |
| | GCTGAAGCTGAAGGGCGGCGGCCACTACCTGGT | |
| | GGAGTTCAAGTCAATCTACATGGCCAAGAAGCC | |
| | CGTGAAGCTGCCCGGCTACTACTACGTGGACTCC | |
| | AAGCTGGACATCACCTCCCACAACGAGGACTAC | |
| | ACCGTGGTGGAGCAGTACGAGCGCGCCGAGGCC | |
| | CGCCACCACCTGTTCCAGTAGGCTGCCTTCTGCG | |
| | GGGCTTGCCTTCTGGCCATGCCCTTCTTCTCTCCC | |
| | TTGCACCTGTACCTCTTGGTCTTTGAATAAAGCC | |
| | TGAGTAGGAAGTGAGGGTCTAGAACTAGTGTCG | |
| | ACGCAAATCAGTTCTGGACCAGCGAGCTGTGCTG | |
| | CGACTCGTGGCGTAATCATGGTCATAGCTGTTTC | |
| | CTGTGTGAAATTGTTATCCGCTCACAATTCCACA | |
| | CAACATACGAGCCGGAAGCATAAAGTGTAAAGC | |
| | CTGGGGTGCCTAATGAGTGAGCTAACTCACATTA | |
| | ATTGCGTTGCGCTCACTGCCCGCTTTCCAGTCGG | |
| | GAAACCTGTCGTGCCAGCTGCATTAATGAATCGG | |
| | CCAACGCGCGGGGAGAGGCGGTTTGCGTATTGG | |
| | GCGCTCTTCCGCTTCCTCGCTCACTGACTCGCTGC | |
| | GCTCGGTCGTTCGGCTGCGGCGAGCGGTATCAGC | |
| | TCACTCAAAGGCGGTAATACGGTTATCCACAGA | |
| | ATCAGGGGATAACGCAGGAAAGAACATGTGAGC | |
| | AAAAGGCCAGCAAAAGGCCAGGAACCGTAAAA | |
| | AGGCCGCGTTGCTGGCGTTTTTCCATAGGCTCCG | |
| | CCCCCCTGACGAGCATCACAAAAATCGACGCTC | |
| | AAGTCAGAGGTGGCGAAACCCGACAGGACTATA | |
| | AAGATACCAGGCGTTTCCCCCTGGAAGCTCCCTC | |
| | GTGCGCTCTCCTGTTCCGACCCTGTCGCTTACCG | |
| | GATACCTGTCCGCCTTTCTCCCTTCGGGAAGCGT | |
| | GGCGCTTTCTCATAGCTCACGCTGTAGGTATCTC | |
| | AGTTCGGTGTAGGTCGTTCGCTCCAAGCTGGGCT | |
| | GTGTGCACGAACCCCCCGTTCAGCCCGACCGCTG | |
| | CGCCTTATCCGGTAACTATCGTCTTGAGTCCAAC | |
| | CCGGTAAGACACGACTTATCGCCACTGGCAGCA | |
| | GCCACTGGTAACAGGATTAGCAGAGCGAGGTAT | |
| | GTAGGCGGTGCTACAGAGTTCTTGAAGTGGTGGC | |
| | CTAACTACGGCTACACTAGAAGAACAGTATTTGG | |
| | TATCTGCGCTCTGCTGAAGCCAGTTACCTTCGGA | |
| | AAAAGAGTTGGTAGCTCTTGATCCGGCAAACAA | |
| | ACCACCGCTGGTAGCGGTGGTTTTTTTGTTTGCA | |
| | AGCAGCAGATTACGCGCAGAAAAAAAGGATCTC | |
| | AAGAAGATCCTTTGATCTTTTCTACGGGGTCTGA | |
| | CGCTCAGTGGAACGAAAACTCACGTTAAGGGAT | |
| | TTTGGTCATGAGATTATCAAAAAGGATCTTCACC | |
| | TAGATCCTTTTAAATTAAAAATGAAGTTTTAAAT | |
| | CAATCTAAAGTATATATGAGTAAACTTGGTCTGA | |
| | CAGTTACCAATGCTTAATCAGTGAGGCACCTATC | |
| | TCAGCGATCTGTCTATTTCGTTCATCCATAGTTGC | |
| | CTGACTCCCCGTCGTGTAGATAACTACGATACGG | |
| | GAGGGCTTACCATCTGGCCCCAGTGCTGCAATGA | |
| | TACCGCGAGACCCACGCTCACCGGCTCCAGATTT | |
| | ATCAGCAATAAACCAGCCAGCCGGAAGGGCCGA | |
| | GCGCAGAAGTGGTCCTGCAACTTTATCCGCCTCC | |
| | ATCCAGTCTATTAATTGTTGCCGGGAAGCTAGAG | |
| | TAAGTAGTTCGCCAGTTAATAGTTTGCGCAACGT | |
| | TGTTGCCATTGCTACAGGCATCGTGGTGTCACGC | |
| | TCGTCGTTTGGTATGGCTTCATTCAGCTCCGGTTC | |
| | CCAACGATCAAGGCGAGTTACATGATCCCCCATG | |
| | TTGTGCAAAAAAGCGGTTAGCTCCTTCGGTCCTC | |
| | CGATCGTTGTCAGAAGTAAGTTGGCCGCAGTGTT | |
| | ATCACTCATGGTTATGGCAGCACTGCATAATTCT | |
| | CTTACTGTCATGCCATCCGTAAGATGCTTTTCTGT | |
| | GACTGGTGAGTACTCAACCAAGTCATTCTGAGAA | |
| | TAGTGTATGCGGCGACCGAGTTGCTCTTGCCCGG | |
| | CGTCAATACGGGATAATACCGCGCCACATAGCA | |
| | GAACTTTAAAAGTGCTCATCATTGGAAAACGTTC | |
| | TTCGGGGCGAAAACTCTCAAGGATCTTACCGCTG | |
| | TTGAGATCCAGTTCGATGTAACCCACTCGTGCAC | |
| | CCAACTGATCTTCAGCATCTTTTACTTTCACCAGC | |
| | GTTTCTGGGTGAGCAAAAACAGGAAGGCAAAAT | |
| | GCCGCAAAAAAGGGAATAAGGGCGACACGGAA | |
| | ATGTTGAATACTCATACTCTACCTTTTTCAATATT | |
| | ATTGAAGCATTTATCAGGGTTATTGTCTCATGAG | |
| | CGGATACATATTTGAATGTATTTAGAAAAATAAA | |
| | CAAATAGGGGTTCCGCGCACATTTCCCCGAAAA | |
| | GTGCCACCTGACGTCTAAGAAACCATTATTATCA | |
| | TGACATTAACCTATAAAAATAGGCGTATCACGA | |
| | GGCCCTTTCGTC | |
| pOVA | gatttataagggattttgccgatttcggcctattggttaaaaaatgagctgatttaac | 3 |
| | aaaaatttaacgcgaattaattctgtggaatgtgtgtcagttagggtgtggaaagtc | |
| | cccaggctccccagcaggcagaagtatgcaaagcatgcatctcaattagtcagc | |
| | aaccaggtgtggaaagtccccaggctccccagcaggcagaagtatgcaaagc | |
| | atgcatctcaattagtcagcaaccatagtcccgcccctaactccgcccatcccgc | |
| | ccctaactccgcccagttccgcccattctccgccccatggctgactaattttttttatt | |
| | tatgcagaggccgaggccgcctctgcctctgagctattccagaagtagtgagga | |
| | ggcttttttggaggcctaggcttttgcaaaaagctcccgggagcttgtatatccattt | |
| | tcggatctgatcaagagacaggatgaggatcgtttcgcatgattgaacaagatgg | |
| | attgcacgcaggttctccggccgcttgggtggagaggctattcggctatgactgg | |
| | gcacaacagacaatcggctgctctgatgccgccgtgttccggctgtcagcgcag | |
| | gggcgcccggttctttttgtcaagaccgacctgtccggtgccctgaatgaactgc | |
| | aggacgaggcagcgcggctatcgtggctggccacgacgggcgttccttgcgc | |
| | agctgtgctcgacgttgtcactgaagcgggaagggactggctgctattgggcga | |
| | agtgccggggcaggatctcctgtcatctcaccttgctcctgccgagaaagtatcc | |
| | atcatggctgatgcaatgcggcggctgcatacgcttgatccggctacctgcccat | |
| | tcgaccaccaagcgaaacatcgcatcgagcgagcacgtactcggatggaagc | |
| | cggtcttgtcgatcaggatgatctggacgaagagcatcaggggctcgcgccag | |
| | ccgaactgttcgccaggctcaaggcgcgcatgcccgacggcgaggatctcgtc | |
| | gtgacccatggcgatgcctgcttgccgaatatcatggtggaaaatggccgcttttc | |
| | tggattcatcgactgtggccggctgggtgtggcggaccgctatcaggacatagc | |
| | gttggctacccgtgatattgctgaagagcttggcggcgaatgggctgaccgcttc | |
| | ctcgtgctttacggtatcgccgctcccgattcgcagcgcatcgccttctatcgcctt | |
| | cttgacgagttcttctgagcgggactctggggttcgaaatgaccgaccaagcga | |
| | cgcccaacctgccatcacgagatttcgattccaccgccgccttctatgaaaggtt | |
| | gggcttcggaatcgttttccgggacgccggctggatgatcctccagcgcgggg | |
| | atctcatgctggagttcttcgcccaccccaacttgtttattgcagcttataatggttac | |
| | aaataaagcaatagcatcacaaatttcacaaataaagcatttttttcactgcattcta | |
| | gttgtggtttgtccaaactcatcaatgtatcttatcatgtctgtataccgtcgacctct | |
| | agctagagcttggcgtaatcatggtcatagctgtttcctgtgtgaaattgttatccgc | |
| | tcacaattccacacaacatacgagccggaagcataaagtgtaaagcctggggtg | |
| | cctaatgagtgagctaactcacattaattgcgttgcgctcactgcccgctttccagt | |
| | cgggaaacctgtcgtgccagctgcattaatgaatcggccaacgcgcggggaga | |
| | ggcggtttgcgtattgggcgctcttccgcttcctcgctcactgactcgctgcgctc | |
| | ggtcgttcggctgcggcgagcggtatcagctcactcaaaggcggtaatacggtt | |
| | atccacagaatcaggggataacgcaggaaagaacatgtgagcaaaaggccag | |
| | caaaaggccaggaaccgtaaaaaggccgcgttgctggcgtttttccataggctc | |
| | cgcccccctgacgagcatcacaaaaatcgacgctcaagtcagaggtggcgaaa | |
| | cccgacaggactataaagataccaggcgtttccccctggaagctccctcgtgcg | |
| | ctctcctgttccgaccctgccgcttaccggatacctgtccgcctttctcccttcggg | |
| | aagcgtggcgctttctcatagctcacgctgtaggtatctcagttcggtgtaggtcgt | |
| | tcgctccaagctgggctgtgtgcacgaaccccccgttcagcccgaccgctgcg | |
| | ccttatccggtaactatcgtcttgagtccaacccggtaagacacgacttatcgcca | |
| | ctggcagcagccactggtaacaggattagcagagcgaggtatgtaggcggtgc | |
| | tacagagttcttgaagtggtggcctaactacggctacactagaagaacagtatttg | |
| | gtatctgcgctctgctgaagccagttaccttcggaaaaagagttggtagctcttga | |
| | tccggcaaacaaaccaccgctggtagcggtggtttttttgtttgcaagcagcagat | |
| | tacgcgcagaaaaaaaggatctcaagaagatcctttgatcttttctacggggtctg | |
| | acgctcagtggaacgaaaactcacgttaagggattttggtcatgagattatcaaaa | |
| | aggatcttcacctagatccttttaaattaaaaatgaagttttaaatcaatctaaagtat | |
| | atatgagtaaacttggtctgacagttaccaatgcttaatcagtgaggcacctatctc | |
| | agcgatctgtctatttcgttcatccatagttgcctgactccccgtcgtgtagataact | |
| | acgatacgggagggcttaccatctggccccagtgctgcaatgataccgcgaga | |
| | cccacgctcaccggctccagatttatcagcaataaaccagccagccggaaggg | |
| | ccgagcgcagaagtggtcctgcaactttatccgcctccatccagtctattaattgtt | |
| | gccgggaagctagagtaagtagttcgccagttaatagtttgcgcaacgttgttgc | |
| | cattgctacaggcatcgtggtgtcacgctcgtcgtttggtatggcttcattcagctc | |
| | cggttcccaacgatcaaggcgagttacatgatcccccatgttgtgcaaaaaagc | |
| | ggttagctccttcggtcctccgatcgttgtcagaagtaagttggccgcagtgttatc | |
| | actcatggttatggcagcactgcataattctcttactgtcatgccatccgtaagatg | |
| | cttttctgtgactggtgagtactcaaccaagtcattctgagaatagtgtatgcggcg | |
| | accgagttgctcttgcccggcgtcaatacgggataataccgcgccacatagcag | |
| | aactttaaaagtgctcatcattggaaaacgttcttcggggcgaaaactctcaagga | |
| | tcttaccgctgttgagatccagttcgatgtaacccactcgtgcacccaactgatctt | |
| | cagcatcttttactttcaccagcgtttctgggtgagcaaaaacaggaaggcaaaat | |
| | gccgcaaaaaagggaataagggcgacacggaaatgttgaatactcatactcttc | |
| | ctttttcaatattattgaagcatttatcagggttattgtctcatgagcggatacatattt | |
| | gaatgtatttagaaaaataaacaaataggggttccgcgcacatttccccgaaaag | |
| | tgccacctgacgtcgacggatcgggagatctcccgatcccctatggtgcactctc | |
| | agtacaatctgctctgatgccgcatagttaagccagtatctgctccctgcttgtgtg | |
| | ttggaggtcgctgagtagtgcgcgagcaaaatttaagctacaacaaggcaaggc | |
| | ttgaccgacaattgcatgaagaatctgcttagggttaggcgttttgcgctgcttcgc | |
| | gatgtacgggccagatatacgcgttgacattgattattgactagttattaatagtaat | |
| | caattacggggtcattagttcatagcccatatatggagttccgcgttacataactta | |
| | cggtaaatggcccgcctggctgaccgcccaacgacccccgcccattgacgtca | |
| | ataatgacgtatgttcccatagtaacgccaatagggactttccattgacgtcaatg | |
| | ggtggagtatttacggtaaactgcccacttggcagtacatcaagtgtatcatatgc | |
| | caagtacgccccctattgacgtcaatgacggtaaatggcccgcctggcattatgc | |
| | ccagtacatgaccttatgggactttcctacttggcagtacatctacgtattagtcatc | |
| | gctattaccatggtgatgcggttttggcagtacatcaatgggcgtggatagcggtt | |
| | tgactcacggggatttccaagtctccaccccattgacgtcaatgggagtttgttttg | |
| | gcaccaaaatcaacgggactttccaaaatgtcgtaacaactccgccccattgacg | |
| | caaatgggcggtaggcgtgtacggtgggaggtctatataagcagagctctctgg | |
| | ctaactagagaacccactgcttactggcttatcgaaattaatacgactcactatag | |
| | ggagacccaagcttggtaccgagctcggatccactagtaacggccgccagtgt | |
| | gctggaattcctcgaaagacaactcagagttcaccatgggctccatcggtgcag | |
| | caagcatggaattttgttttgatgtattcaaggagctcaaagtccaccatgccaatg | |
| | agaacatcttctactgccccattgccatcatgtcagctctagccatggtatacctgg | |
| | gtgcaaaagacagcaccaggacacaaataaataaggttgttcgctttgataaact | |
| | tccaggattcggagacagtattgaagctcagtgtggcacatctgtaaacgttcact | |
| | cttcacttagagacatcctcaaccaaatcaccaaaccaaatgatgtttattcgttca | |
| | gccttgccagtagactttatgctgaagagagatacccaatcctgccagaatacttg | |
| | cagtgtgtgaaggaactgtatagaggaggcttggaacctatcaactttcaaacag | |
| | ctgcagatcaagccagagagctcatcaattcctgggtagaaagtcagacaaatg | |
| | gaattatcagaaatgtccttcagccaagctccgtggattctcaaactgcaatggttc | |
| | tggttaatgccattgtcttcaaaggactgtgggagaaagcatttaaggatgaagac | |
| | acacaagcaatgcctttcagagtgactgagcaagaaagcaaacctgtgcagatg | |
| | atgtaccagattggtttatttagagtggcatcaatggcttctgagaaaatgaagatc | |
| | ctggagcttccatttgccagtgggacaatgagcatgttggtgctgttgcctgatga | |
| | agtctcaggccttgagcagcttgagagtataatcaactttgaaaaactgactgaat | |
| | ggaccagttctaatgttatggaagagaggaagatcaaagtgtacttacctcgcat | |
| | gaagatggaggaaaaatacaacctcacatctgtcttaatggctatgggcattactg | |
| | acgtgtttagctcttcagccaatctgtctggcatctcctcagcagagagcctgaag | |
| | atatctcaagctgtccatgcagcacatgcagaaatcaatgaagcaggcagagag | |
| | gtggtagggtcagcagaggctggagtggatgctgcaagcgtctctgaagaattt | |
| | agggctgaccatccattcctcttctgtatcaagcacatcgcaaccaacgccgttct | |
| | cttctttggcagatgtgtttccccttaaaaagaagaaagctgaaaaactctgtccct | |
| | tccaacaagacccagagcactgtagtatcaggggtaaaatgaaaagtatgttatc | |
| | tgctgcatccagacttcataaaagctggagcttaatctagagggccctattctata | |
| | gtgtcacctaaatgctagagctcgctgatcagcctcgactgtgccttctagttgcc | |
| | agccatctgttgtttgcccctcccccgtgccttccttgaccctggaaggtgccact | |
| | cccactgtcctttcctaataaaatgaggaaattgcatcgcattgtctgagtaggtgt | |
| | cattctattctggggggtggggggggcaggacagcaagggggaggattggg | |
| | aagacaatagcaggcatgctggggatgcggtgggctctatggcttctgaggcg | |
| | gaaagaaccagctggggctctagggggtatccccacgcgccctgtagcggcg | |
| | cattaagcgcggcgggtgtggtggttacgcgcagcgtgaccgctacacttgcca | |
| | gcgccctagcgcccgctcctttcgctttcttcccttcctttctcgccacgttcgccg | |
| | gctttccccgtcaagctctaaatcgggggctccctttagggttccgatttagtgcttt | |
| | acggcacctcgaccccaaaaaacttgattagggtgatggttcacgtagtgggcc | |
| | atcgccctgatagacggtttttcgccctttgacgttggagtccacgttctttaatagt | |
| | ggactcttgttccaaactggaacaacactcaaccctatctcggtctattctttt | |
| Forward | 5′-TTGGACCCTCGTACAGAAGCTAATACG-3′ | 4 |
| primer- | | |
| pUCIDT- | | |
| AMP_mTagBF | | |
| P2 | | |
| Reverse | 5′-AAG CAA GCT GAC CCT GAA GTT TTT TTT TTT | 5 |
| primer- | TTT TTT TTT TTT TTT TTT TTT TTT TTT TTT TTT | |
| pUCIDT- | TTT TTT TTT TTT TTT TTT TTT TTT TTT TTT TTT | |
| AMP_mTagBF | TTT TTT TTT TTT TTT TTT TTT TTT TTT TTT TTT | |
| P2-siGFP (AS) | TTT TTT TTT TTT CTT CCT ACT CAG GCT TTA | |
| | TTC AAA GAC CA-3′ (Italic: sequence for antisense | |
| | strand of siGFP) | |
| SS siGFP | 5′-GCA AGC UGA CCC UGA AGU U (dT)_{2 or 6}-3′ | 6, 7 |
| Reverse | 5′-AAA ACT TCA GGG TCA GCT TGC TTT TTT TTT | 8 |
| primer- | TTT TTT TTT TTT TTT TTT TTT TTT TTT TTT TTT | |
| pUCIDT- | TTT TTT TTT TTT TTT TTT TTT TTT TTT TTT TTT | |
| AMP_mTagBF | TTT TTT TTT TTT TTT TTT TTT TTT TTT TTT TTT | |
| P2-siGFP (SS) | TTT TTT TTT TTT CTT CCT ACT CAG GCT TTA | |
| | TTC AAA GAC CA-3′ | |
| | (Italic: sequence for sense strand of siGFP) | |
| AS siGFP | 5′-AAC UUC AGG GUC AGC UUG C (dT)_{2 or 6}-3′ | 9, 10 |
| Forward | 5′-TTGGACCCTCGTACAGAAGCTAATACG-3′ | 4 |
| primer- | | |
| pUCIDT- | | |
| AMP_DsRed | | |
| Express2 RFP | | |
| Reverse | 5′-AAG CAA GCT GAC CCT GAA GTT TTT TTT TTT | 5 |
| primer- | TTT TTT TTT TTT TTT TTT TTT TTT TTT TTT TTT | |
| pUCIDT- | TTT TTT TTT TTT TTT TTT TTT TTT TTT TTT TTT | |
| AMP_DsRed | TTT TTT TTT TTT TTT TTT TTT TTT TTT TTT TTT | |
| Express2 RFP- | TTT TTT TTT TTT CTT CCT ACT CAG GCT TTA | |
| siGFP (AS) | TTC AAA GAC CA-3′ (Italic: sequence for antisense | |
| | strand of siGFP) | |
| SS siGFP | 5′-GCA AGC UGA CCC UGA AGU U (dT)_{2 or 6}-3′ | 6, 7 |
| Reverse | 5′-AAA ACT TCA GGG TCA GCT TGC TTT TTT TTT | 8 |
| primer- | TTT TTT TTT TTT TTT TTT TTT TTT TTT TTT TTT | |
| pUCIDT- | TTT TTT TTT TTT TTT TTT TTT TTT TTT TTT TTT | |
| AMP_DsRed | TTT TTT TTT TTT TTT TTT TTT TTT TTT TTT TTT | |
| Express2 RFP- | TTT TTT TTT TTT CTT CCT ACT CAG GCT TTA | |
| siGFP (SS) | TTC AAA GAC CA-3′ | |
| | (Italic: sequence for sense strand of siGFP) | |
| AS siGFP | 5′-AAC UUC AGG GUC AGC UUG C (dT)_{2 or 6}-3′ | 9, 10 |
| Forward | 5′-GAGAACCCACTGCTTACTGG-3′ | 11 |
| primer-pOVA | | |
| Reverse | 5′-AAA GTA GGT GCA GGA ACT GTG TTT TTT TTT | 12 |
| primer- | TTT TTT TTT TTT TTT TTT TTT TTT TTT TTT TTT | |
| pOVA- | TTT TTT TTT TTT TTT TTT TTT TTT TTT TTT TTT | |
| siSTAT3 (AS) | TTT TTT TTT TTT TTT TTT TTT TTT TTT TTT TTT | |
| | TTT TTT TTT TTT TCA GCG AGC TCT AGC ATT | |
| | TAG G-3′ | |
| | (Italic: sequence for antisense strand of siSTAT3) | |
| SS siSTAT3 | 5′-AGU AGG UGC AGG AAC UGU G (dT)_{2 or 6}-3′ | 13, 14 |
| Reverse | 5′-AAC ACA GTT CCT GCA CCT ACT TTT TTT TTT | 15 |
| primer- | TTT TTT TTT TTT TTT TTT TTT TTT TTT TTT TTT | |
| POVA- | TTT TTT TTT TTT TTT TTT TTT TTT TTT TTT TTT | |
| siSTAT3 (SS) | TTT TTT TTT TTT TTT TTT TTT TTT TTT TTT TTT | |
| | TTT TTT TTT TTT TCA GCG AGC TCT AGC ATT | |
| | TAG G-3′ | |
| | (Italic: sequence for sense strand of siSTAT3) | |
| AS siSTAT3 | 5′-CAC AGU UCC UGC ACC UAC U (dT) 2 or 6-3′ | 16, 17 |

In the mRNA-siRNA according to the present invention, the double-stranded hybrid formed between the poly (A) of mRNA and the poly (T) of siRNA was cleaved by RNase H in an intracellular environment to be separated into mRNA capable of expressing a target protein and siRNA capable of inhibiting the expression of a specific gene, and as a result, simultaneous expression and inhibition of a protein was enabled in a subject (FIG. 4).

Meanwhile, as illustrated in FIG. 4, the mRNA-siRNA according to the present invention was characterized in that one or more siRNAs may be linked to the 3′ end of mRNA. At this time, from the second siRNA after the introduction of the first siRNA, the experiment was performed under the same conditions as the annealing, but a siRNA sequence was added to the reverse primer. The siRNA to be added may be introduced and added as many siRNAs as the number to be introduced with respect to the reverse primer. For example, all primers used to introduce the second siRNA were shown in Table 4 below.

TABLE 4

| Gene name | Sequence information | SEQ ID NO: |
|---|---|---|
| Forward primer- | TTGGACCCTCGTACAGAAGCTAATACG | 4 |
| pUCIDT- | | |
| AMP_mTagBFP2 | | |
| Reverse primer- | AAG CAA GCT GAC CCT GAA GTT AAAA | 18 |
| pUCIDT- | AAG CAA GCT GAC CCT GAA GTT TTT TTT | |
| AMP_mTagBFP2- | TTT TTT TTT TTT TTT TTT TTT TTT TTT | |
| siGFP (two) (AS) | TTT TTT TTT TTT TTT TTT TTT TTT TTT | |
| | TTT TTT TTT TTT TTT TTT TTT TTT TTT | |
| | TTT TTT TTT TTT TTT TTT TTT TTT TTT | |
| | TTT TTT CTT CCT ACT CAG GCT TTA TTC | |
| | AAA GAC CA (Italic: sequence for antisense | |
| | strand of two siGFPs) | |
| Reverse primer- | AAA ACT TCA GGG TCA GCT TGC AAAA | 19 |
| pUCIDT- | AAA ACT TCA GGG TCA GCT TGC TTT TTT | |
| AMP_mTagBFP2- | TTT TTT TTT TTT TTT TTT TTT TTT TTT | |
| siGFP (two) (SS) | TTT TTT TTT TTT TTT TTT TTT TTT TTT | |
| | TTT TTT TTT TTT TTT TTT TTT TTT TTT | |
| | TTT TTT TTT TTT TTT TTT TTT TTT TTT | |
| | TTT TTT CTT CCT ACT CAG GCT TTA TTC | |
| | AAA GAC CA (Italic: sequence for sense | |
| | strand of two siGFPs) | |
| AS siGFP | 5′-AAC UUC AGG GUC AGC UUG C (dT)_{2 or 6}-3′ | 9, 10 |
| SS siGFP | 5′-GCA AGC UGA CCC UGA AGU U (dT)_{2 or 6}-3′ | 6, 7 |

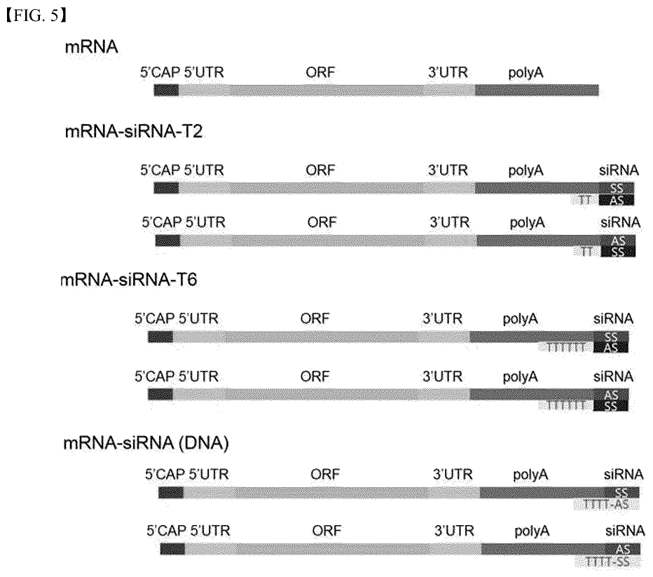

In Example, the present inventors evaluated the protein expression efficiency of mRNA and the gene expression inhibition ability of siRNA using siRNA-Oligo (dT), in which a sense or antisense sequence of siRNA and oligo (dT) of different lengths were combined in various configurations as follows, and intended to derive an optimal structure of a nucleic acid construct with excellent gene expression and inhibition effects (FIG. 5).
5′-siRNA Sequence (Antisense/Sense) Complementarily Binding to siGFP-dTdT-3′ (T2)
5′—siRNA sequence (antisense sense) complementarily binding to siGFP-dTdTdTdTdTdT-3′ (T6)
5′—DNA sequence (antisense sense) complementarily binding to siGFP-dTdTdTdT-3′ (DNA)

### Experimental Example 1. Identification of Each Reaction Product

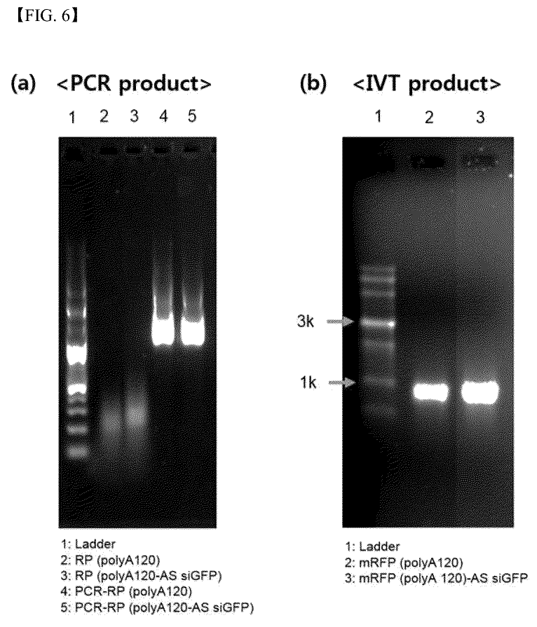

After each reaction in Example was completed, the reaction product was electrophoresed on a 1% agarose gel at 100 V for 30 minutes, and the PCR reaction product and the in vitro transcription product were confirmed, and then results were illustrated in FIG. 6.

As illustrated in FIG. 6A, the sizes of DNA fragments amplified using the reverse primer were confirmed as 971 bp (poly (A) 120, Lane 4) and 992 bp (poly (A) 120-AS siGFP, Lane 5), respectively. Through the results, it was confirmed that the DNA template was effectively amplified through a reverse primer designed to introduce the poly (T) tail and the siRNA sequence to 3′ next to the 3′ UTR by performing the PCR.

In addition, as illustrated in FIG. 6B, as a result of performing in vitro transcription using the DNA template, it was confirmed that mRFP (Lane 2) in which a poly (A) 120 sequence was introduced to the 3′ end and mRFP (Lane 3) in which a poly (A) 120-AS siGFP sequence was introduced to the 3′ end were synthesized, respectively.

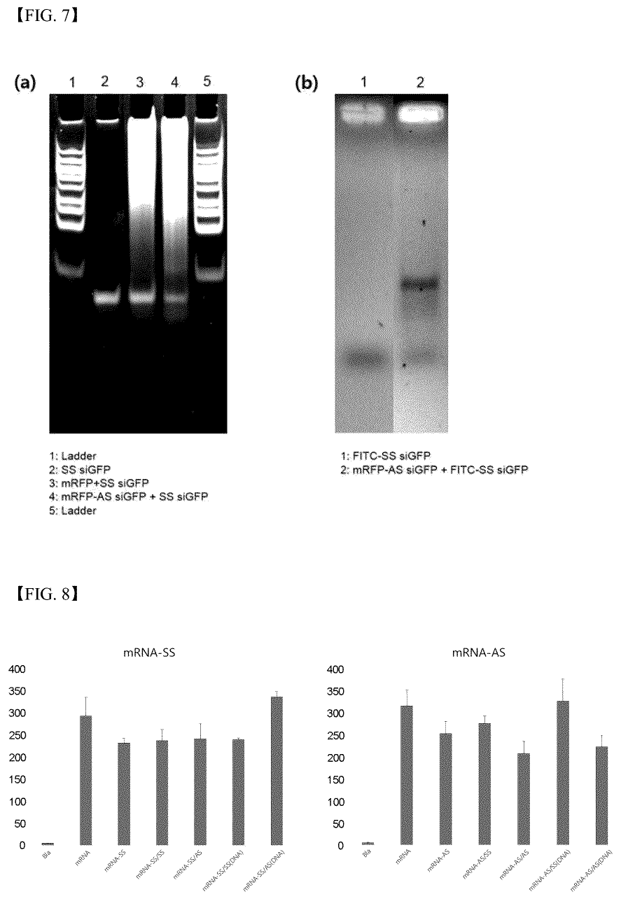

Similarly, the results of confirming the mRNA-siRNA nucleic acid construct prepared by the method described in Example above were illustrated in FIG. 7.

As illustrated in FIG. 7A, as a result of fast-cooling mRFP in which only the poly (A) 120 sequence was introduced to the 3′ end of the mRNA and mRFP in which the poly (A) 120 and antisense siGFP sequences were introduced together to the 3′ end of the mRNA together with the sense siGFP strand, it was confirmed that a band of sense siGFP injected into the mRFP in which the poly (A) 120 and the antisense siGFP sequences were introduced become faded, and thus, it was confirmed that the sense siGFP was successfully annealed to the mRFP introduced with the poly (A) 120 and antisense siGFP sequences.

In addition, as illustrated in FIG. 7B, it was confirmed that when FITC-labeled sense siGFP was annealed to mRFP (mRFP-AS siGFP) into which antisense siGFP was introduced at the 3′ end (mRFP-AS siGFP+FITC-SS siGFP, Lane 2), a band appeared at an upper part as compared to FITC-labeled sense siGFP (FITC-SS siGFP, Lane 1). Through the results, it was confirmed that when mRNA including the siRNA sequence at the 3′ end and siRNA complementarily binding to the siRNA sequence were annealed, an mRNA-siRNA nucleic acid structure partially hybridized with each other was formed.

### Experimental Example 2. Evaluation of Protein Expression Ability of Each Construct

The protein expression effects of different mRNA-siRNA nucleic acid constructs prepared in Examples were confirmed. Specifically, cells were seeded in a 96-well plate in an amount of 1×10⁴ cells per well, and the cells were transfected with each construct at a dose of 10 pg mRNA/cell using a transfection reagent. After 24 hours of transfection, the fluorescence intensity of the red fluorescent protein expressed in each Example was measured using a flow cytometer FACS Calibur (BD Biosciences, CA) to confirm the translation efficiency of each construct, and the results were illustrated in FIG. 8. All Examples used in the experiment were shown in Table 5 below. In Table 5 below, mRNA-SS/SS or mRNA-SS/AS means annealed with the siGFP-T2 sequence, and mRNA-AS/SS (DNA) or mRNA-AS/AS (DNA) means annealed with a sequence in which four additional dT sequences are introduced to DNA having a siGFP sequence.

TABLE 5
Example of the present invention

| mRNA-siRNA(SS or AS) | | siRNA and/or DNA | | |
|---|---|---|---|---|
| (5′-3′) | | (5′-3′) | | |
| mRNA | siRNA | siRNA | Oligo(dT) | Example |
| — | — | — | — | Blank |
| mRFP | — | — | — | mRNA |
| | siGFP(SS) | — | — | mRNA-SS |
| | | siGFP(SS) | TT(T2) | mRNA-SS/SS |
| | | siGFP(AS) | TT(T2) | mRNA-SS/AS |
| | | siGFP(SS) | TTTT(DNA) | mRNA-SS/SS(DNA) |
| | | (DNA) | | |
| | | siGFP(AS) | TTTT(DNA) | mRNA-SS/AS(DNA) |
| | | (DNA) | | |
| | siGFP(AS) | — | — | mRNA-AS |
| | | siGFP(SS) | TT(T2) | mRNA-AS/SS |
| | | siGFP(AS) | TT(T2) | mRNA-AS/AS |
| | | siGFP(SS) | TTTT(DNA) | mRNA-AS/SS(DNA) |
| | | (DNA) | | |
| | | siGFP(AS) | TTTT(DNA) | mRNA-AS/AS(DNA) |
| | | (DNA) | | |

As illustrated in FIG. 8, it was confirmed that the translation efficiency was commonly lowered in Example in which the mRNA including the siRNA sequence at the 3′ end was transfected. On the other hand, in the case of Example in which siGFP (AS)-TTTT (DNA) was annealed to mRNA (SS) and Example in which siGFP (SS)-TTTT (DNA) was annealed to mRNA (AS), it was confirmed that the decreased translation efficiency was significantly increased, and the fluorescence intensity was stronger than that of a case of transfecting mRFP, and the protein expression ability of mRNA was completely restored.

Through the results, it was confirmed that when mRNA including the siRNA (sense/antisense) sequence at the 3′ end was hybridized with siRNA including a siRNA (antisense/sense) sequence complementarily binding to the siRNA and oligo (dT) complementarily binding to the mRNA, the translation efficiency of mRNA inhibited by binding of the siRNA sequence may be restored by a DNA sequence annealed to the siRNA sequence of mRNA-siRNA (SS or AS). This seems to be caused by a mechanism in which the mRNA-siRNA/DNA hybrid construct according to the present invention was cleaved by intracellular RNase H.

In addition, by confirming that the protein translation efficiency was inhibited in the case of siRNA-dTdT (T2) annealed to mRNA-siRNA (SS or AS), it may be seen that the length of the DNA sequence annealed with the mRNA-siRNA (SS or AS) needs to be at least two, so that the siRNA sequence additionally introduced by intracellular RNase His truncated and the mRNA protein translation efficiency may be restored. Accordingly, in subsequent experiments, mRNA-siRNA (SS or AS) was annealed with siRNA-dTdTdTdTdTdT (T6) and separated into mRNA and siRNA by RNase H when introduced into cells, and then designed so that the protein translation efficiency of mRNA and the protein inhibition efficiency of siRNA were maintained.

### Experimental Example 3. Evaluation of Protein Expression Ability and Gene Expression Inhibition Ability of Each Construct

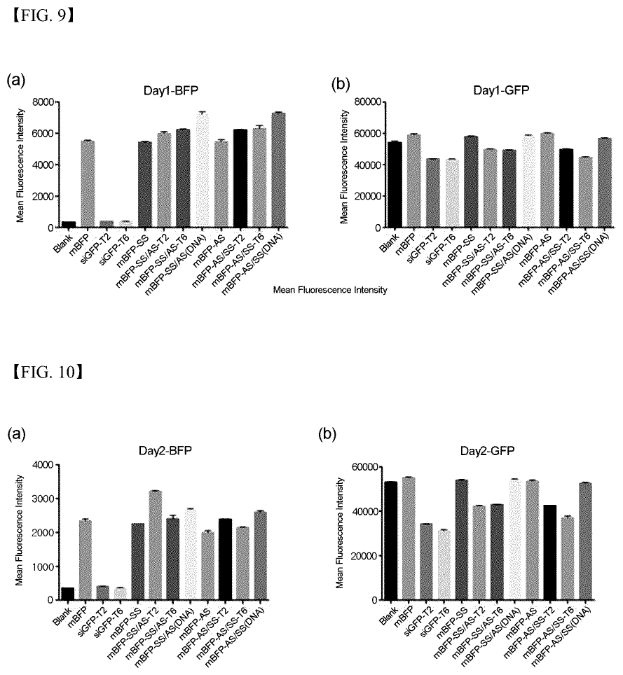

The protein expression ability and gene expression inhibition effects of the different mRNA-siRNA constructs prepared in Examples were confirmed. Specifically, cells were seeded in a 96-well plate in an amount of 1×10⁴ cells per well, and the cells were transfected with each construct at a dose of 10 pg mRNA/cell using a transfection reagent. After 24 and 48 hours of transfection, the fluorescence intensities of a blue fluorescent protein and a green fluorescent protein expressed in each Example were measured using a flow cytometer FACS Calibur (BD Biosciences, CA) to confirm the protein expression efficiency of each construct, and the results were illustrated in FIGS. 9 and 10. All Examples used in the experiment were shown in Table 6 below.

TABLE 6
As illustrated in FIG. 9A, it was confirmed that the blue fluorescence intensity
was reduced in Example of transfecting the mRNA including the siRNA

| mRNA-siRNA(SS or AS) | | siRNA and/or DNA | | |
|---|---|---|---|---|
| (5′-3′) | | (5′-3′) | | |
| mRNA | siRNA | siRNA | Oligo(dT) | Example |
| — | — | — | — | Blank |
| mBFP | — | — | — | mBFP |
| — | — | siGFP | TT(T2) | siGFP-T2 |
| — | — | siGFP | TTTTTT(T6) | siGFP-T6 |
| mBFP | siGFP(SS) | — | — | mBFP-SS |
| | | siGFP(AS) | TT(T2) | mBFP-SS/AS-T2 |
| | | siGFP(AS) | TTTTTT(T6) | mBFP-SS/AS-T6 |
| | | siGFP(AS) | TTTT(DNA) | mBFP-SS/AS(DNA) |
| | siGFP(AS) | — | — | mBFP-AS |
| | | siGFP(SS) | TT(T2) | mBFP-AS/SS-T2 |
| | | siGFP(SS) | TTTTTT(T6) | mBFP-AS/SS-T6 |
| | | siGFP(SS) | TTTT(DNA) | mBFP-AS/SS(DNA) |

sequence at the 3′ end, and through this, the protein expression efficiency was lowered by linking the siRNA sequence. On the other hand, in the case of Example in which siGFP (AS)-TTTTTT (T6) and siGFP (AS)-TTTT (DNA) were annealed to mBFP (SS) and Example in which siGFP (SS)-TTTTTT (T6) and siGFP (SS)-TTTT (DNA) were annealed to mBFP (AS), it was confirmed that the decreased translation efficiency was significantly increased, and the fluorescence intensity was stronger than that of a case of transfecting mBFP, and the protein expression ability of mRNA was completely restored.

Through the result, it was confirmed that in the case of hybridizing mRNA including the siRNA (sense/antisense) sequence at the 3′ end and siRNA including the siRNA (antisense/sense) sequence complementarily binding to the siRNA (mRNA-siRNA), the decrease in translation efficiency may be overcome by linking the siRNA sequence to mRNA. Furthermore, it was confirmed that the decreased translation efficiency was significantly increased when the oligo (dT) located at the 3′ end of siRNA to be annealed was T6 rather than T2, and T4 rather than T6.

Meanwhile, as illustrated in FIG. 9B, it was confirmed that in Example in which siGFP (AS)-TTTTTT (T6) was annealed to mBFP (SS) and Example in which siGFP (SS)-TTTTTT (T6) was annealed to mBFP (AS), the green fluorescence intensity was decreased, especially the measured fluorescence intensity was similar to that of a case of transfecting normal siRNA.

Through the result, it was confirmed that independently of mRNA protein expression, in the case of hybridizing mRNA including the siRNA (sense/antisense) sequence at the 3′ end and siRNA including the siRNA (antisense/sense) sequence complementarily binding to the siRNA (mRNA-siRNA), the expression of a gene targeted by the siRNA was significantly inhibited. In particular, it was confirmed that the protein expression inhibition efficiency was highest when the oligo (dT) located at the 3′ end of the annealed siRNA was T6.

It was confirmed that these results were similar even after 48 hours of transfection. However, as illustrated in FIG. 10A, the reduced translation efficiency by linking the siRNA sequence to the mRNA was most greatly increased when the oligo (dT) located at the 3′ end of the siRNA to be annealed was T2 or T4. In addition, as illustrated in FIG. 10B, the gene expression inhibition efficiency was the highest in Example in which siGFP (SS)-TTTTTT (T6) was annealed to mBFP (AS).

As the above-described analysis result, it was confirmed that the mRNA-siRNA nucleic acid construct according to the present invention was simultaneously introduced into cells with high efficiency, and then a double-stranded hybrid formed between mRNA and siRNA was cleaved by intracellular RNase to be separated into mRNA capable of expressing a target protein and siRNA capable of inhibiting the expression of a specific gene. Furthermore, it was confirmed that by having such a specific structure, the mRNA-siRNA nucleic acid construct according to the present invention may successfully achieve simultaneously expression of a target protein by mRNA and inhibition of the expression of a specific gene by siRNA in one cell.

### Experimental Example 4. Evaluation of Immunogenicity Enhancing Ability of Nucleic Acid Construct According to the Present Invention

It is known that mRNA has an effect of inducing an immune response by stimulating a toll-like receptor (TLR). That is, mRNA itself has an immune response-inducing effect and may express an antigen (protein) at the same time, and thus may be used as a vaccine without a separate adjuvant. Meanwhile, such an mRNA-based vaccine expresses a specific antigen in dendritic cells or T cells, and at this time, the STAT3 protein inhibits the activation of the immune response, thereby inhibiting the maturation of dendritic cells.

Accordingly, the present inventors determined that when a siRNA sequence that inhibits the expression of STAT3 was introduced into an mRNA construct designed to express an antigen by using the nucleic acid construct according to the present invention, the maturation of dendritic cells may be effectively promoted at the same time without interfering with immune escape, and performed the experiment as follows. First, in the same manner as described in Example, mRNA expressing ovalbumin (OVA) and siRNA inhibiting the expression of the STAT3 gene were introduced into the nucleic acid construct according to the present invention. As a comparative experimental group, siRNA (siSTAT3) targeting STAT3 and a simple mixture (mOVA+siSTAT3) of ovalbumin-expressing mRNA and siSTAT3 were used. All mRNA and siRNA sequences used in the experiment were shown in Table 3 in Example.

### 4-1. STAT3 Protein Expression Inhibitory Effect

Bone marrow-derived dendritic cells (BMDCs) were treated with the prepared siSTAT3, a simple mixture of mOVA and siSTAT3, and a mOVA-siSTAT3/siSTAT3 construct, respectively. Specifically, BMDCs (1×10⁶ cells/well) were cultured in X-vivo 15 media containing 10% FBS and antibiotics (100 unit/ml penicillin and 100 μg/ml streptomycin) in a 24-well plate at 37° C. for 24 hours. The cultured BMDCs were transfected with siSTAT3, mOVA+siSTAT3, and mOVA-siSTAT3/siSTAT3, respectively. The expression level of the STAT3 gene in each BMDC was analyzed using a qRT-PCR method, and the results were shown in FIG. 11.

As illustrated in FIG. 11, it was confirmed that when the nucleic acid construct according to the present invention was treated with mOVA-siSTAT3/siSTAT3 (mOVA-T6-STAT3i) in which mOVA and siSTAT3 were introduced, the expression of STAT3 was significantly inhibited compared to mOVA+siSTAT3 in which mOVA and siSTAT3 were simply mixed. Through the results, it was confirmed that the effect of improving immunogenicity was much better when the mRNA-siRNA nucleic acid construct in the form of a combination of mRNA and siRNA for protein expression was administered than when the separated mRNA and siRNA were mixed and administered.

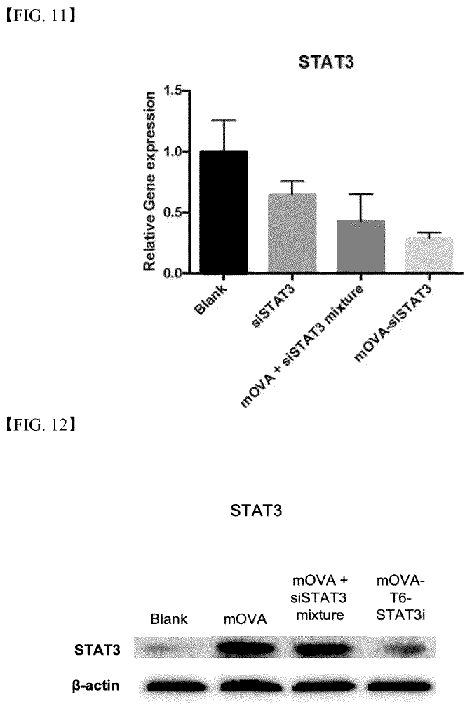

In addition, the confirmation result of the expression change was confirmed through western blot, and the results were shown in FIG. 12.

As may be seen in FIG. 12, it was confirmed that the immunogenicity improvement effect was very excellent when the mRNA-siRNA nucleic acid construct in the form of the combination of mRNA and siRNA was administered.

### 4-2. Dendritic Cell Maturation Promoting Effect

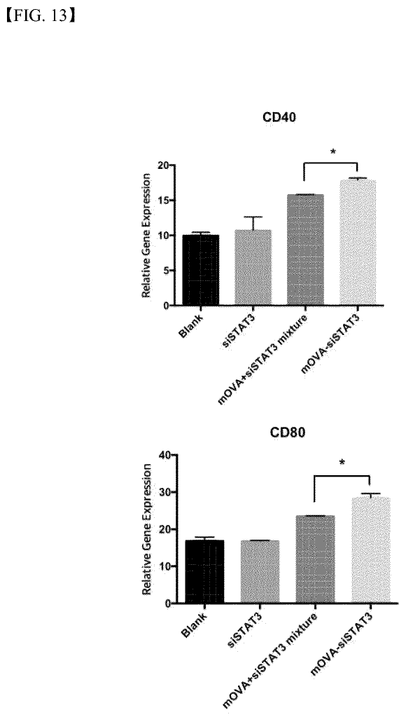

In the same manner as described in Experimental Example 4-1, bone marrow-derived dendritic cells (BMDCs) were treated with the prepared siSTAT3, mOVA+siSTAT3, and mOVA-siSTAT3/siSTAT3 constructs, respectively, and incubated for 12 hours to 48 hours. Some cells were taken and the expression levels of CD40 and CD80 markers expressed on the cell surface were analyzed by flow cytometry (Fluorescence-activated cell sorting, FACS). The results were shown in FIG. 13.

As illustrated in FIG. 13, it was confirmed that when the nucleic acid construct according to the present invention was treated with mOVA-siSTAT3/siSTAT3 in which mOVA and siSTAT3 were introduced, the expression of both CD40 and CD80, which were phenotypes of mature dendritic cells, was remarkably increased compared to mOVA+siSTAT3 in which mOVA and siSTAT3 were simply mixed. Through the results, it was confirmed that the effect of promoting the dendritic cell maturation was much better when the mRNA-siRNA nucleic acid construct in the form of a combination of mRNA and siRNA for protein expression was administered than when the separated mRNA and siRNA were mixed and administered.

### 4-3. Confirmation of Ovalbumin (OVA) Expression

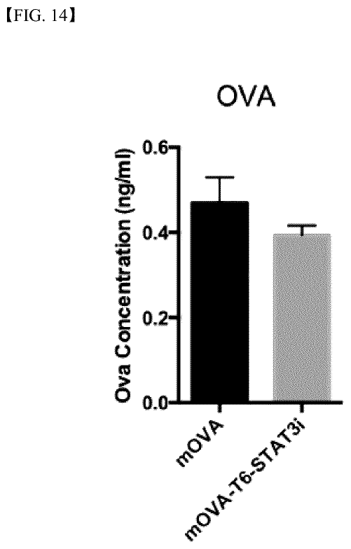

Dendritic cells were treated with a mOVA-siSTAT3/siSTAT3 (mOVA-T6-STAT3i) construct, and then the expression level of OVA was measured by ELISA, and the results were illustrated in FIG. 14.

As may be seen in FIG. 14, a significant amount of OVA expression was confirmed through the treatment of the mOVA-siSTAT3/siSTAT3 construct.

### Experimental Example 5. Tetramer Assay

For in vivo studies, a mOVA+siSTAT3 mixture or mOVA-siSTAT3/siSTAT3 (mOVA-T6-STAT3i) was formulated into lipid nanoparticles by rapid mixing of the following compositions using a microfluidic device (NanoAssemblr Benchtop (Precision Nanosystems, Canada)): Ethanol phase containing a lipid mixture (C₁₂₋₂₀₀:dioleoyl phosphatidyl ethanolamine (DOPE):cholesterol:C₁₄-polyethylene glycol (PEG) 2000=35:16: 46.5:2.5, molar ratio) and aqueous phase (10 mM citrate buffer, pH 3).

To confirm the induction of cytotoxic T cell immunity, the lipid-formulated mOVA+siSTAT3 mixture or the mOVA-siSTAT3/siSTAT3 nanoparticles were intramuscularly injected into 6-week-old female C57BL/6 mice (20 pmole mOVA+20 pmole siSTAT3 mixture or 20 pmole mOVA-siSTAT3/siSTAT3). After 1 week of injection, splenocytes were isolated and stained with a SIINFEKL (MHC I peptide epitope of OVA) tetramer in combination with an antibody mixture (PerCP/Cyanine5.5-anti-mouse CD8a, FITC-anti-mouse CD44 and APCanti-mouse CD45 (BioLegend, USA)).

The stained cells were validated using a NovoCyte flow cytometer (ACEA), and Flowjo software was used for data analysis.

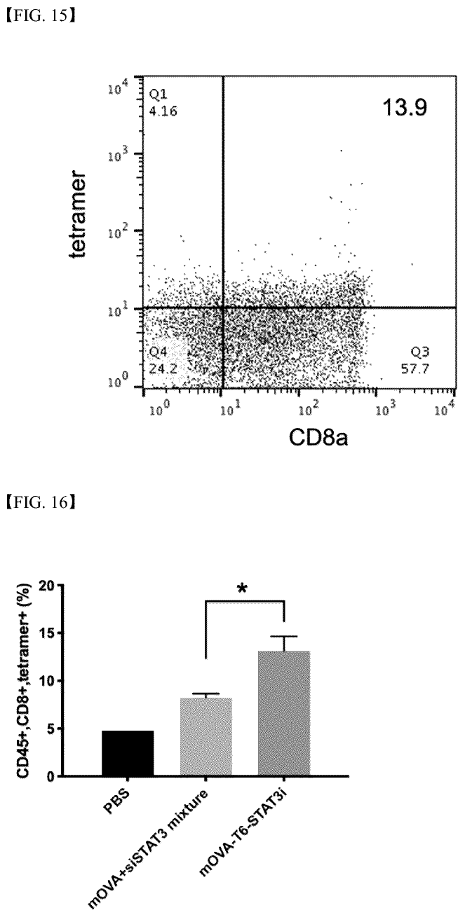

The results were illustrated in FIGS. 15 and 16.

Among the splenocytes, a triple positive cell group of CD45, SIINFEKL and CD8a represented OVA-specific cytotoxic T cells. As illustrated in FIG. 15, 13.9% of splenocytes harvested from mOVA-siSTAT3/siSTAT3 treated dendritic cells were analyzed as a triple positive cell group.

In addition, FIG. 16 illustrates that a higher OVA-specific cytotoxic T cell group was observed in the splenocytes of mice treated with mOVA-siSTAT3/siSTAT3. It was confirmed that improved dendritic cell maturation may be achieved with mRNA having enhanced cytotoxic T cell immunity.

These results exhibit an excellent effect of the nucleic acid construct according to the present invention on a CD8+ T cell inducing effect for an Ovalbumine antigen.

### Experimental Example 6. Total IgG Assay

Analysis of the total IgG content was performed using the mouse set of Experimental Example 5. Serum samples were collected after 1 week of injection. A Nunc Maxisorp immune plate was incubated overnight and coated with 10 μg/ml of ovalbumin (invivogen). The ovalbumin-coated plate was incubated with diluted serum (1:200 in 1% bovine serum albumin (BSA)) at 37° C. for 2 hours, and then washed with a washing buffer (PBS containing 0.05% Tween-20). The washed plate was incubated with a secondary antibody (Mouse IgG heavy and light chain antibodies (Bethyl Laboratories, USA)) for 1 hour, and then washed with a washing buffer, and finally, the plate was treated with a TMB substrate solution (Thermo Scientific) and the reaction was stopped using a stop solution (Thermo Scientific), and then absorbance at 450 nm was measured with a microplate multi-reader.

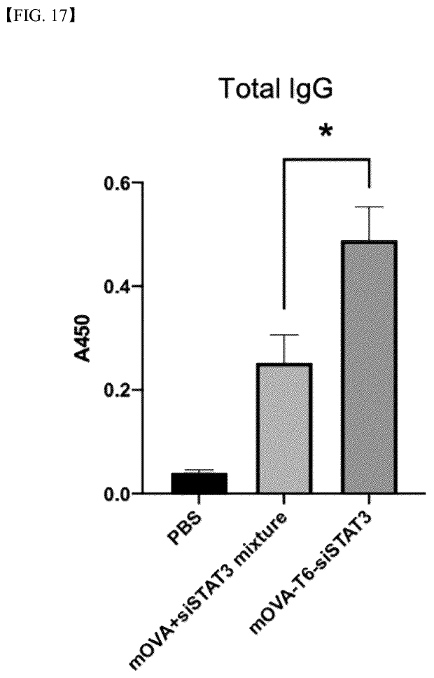

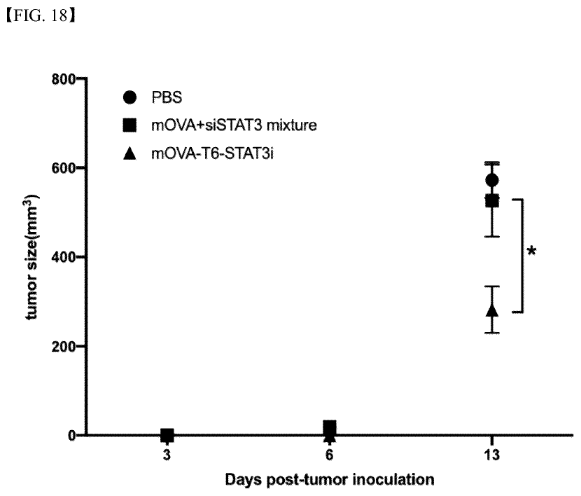

The results were illustrated in FIG. 17.

As may be seen in FIG. 17, it was confirmed that the improvement of OVA-specific IgG expression was induced.

### Experimental Example 7. Confirmation of Reduction of Tumor Size

The tumor inhibition effect was confirmed using the mouse set of Experimental Example 5. After 1 week of drug injection, tumor cells expressing ovalbumin (3107E.G.7-OVA cells) were challenged. After tumor challenge, the tumor size was measured using a digital caliper and the tumor volume was calculated by eq. V=0.5 W² L (V; tumor volume, W; tumor width, L; tumor length).

The results were illustrated in FIG. 18.

As may be seen in FIG. 18, siSTAT3/siSTAT3 (mOVA-T6-STAT3i) exhibited a significantly superior tumor inhibition effect to the simple mixture.

Through the experiments, it was confirmed that in the mRNA-siRNA nucleic acid construct according to the present invention, mRNA and siRNA were introduced into cells at the same time, and mRNA normally expressed a protein to be expressed, and siRNA may specifically inhibit the expression of a specific gene. Through the results, the nucleic acid construct according to the present invention activates an immune system by expressing a target protein in an antigen-presenting cell, and at the same time, overcomes interference of immune escape by blocking the activity of an immune checkpoint protein, and thus may be very usefully used in the fields of immunotherapy, especially in the field of cancer immunotherapy.

1. A nucleic acid construct comprising mRNA and siRNA, comprising:
(i) mRNA comprising a poly (A) tail; and a first siRNA sense or antisense sequence located sequentially from a 5′ direction to a 3′ direction at a 3′ end; and
(ii) a hybridization sequence that complementarily binds to the first siRNA sequence.

2. The nucleic acid construct of claim 1, wherein the hybridization sequence is a second siRNA antisense or sense sequence complementarily binding to the first siRNA sequence; or a DNA antisense or sense sequence complementarily binding to the first siRNA sequence.

3. The nucleic acid construct of claim 1, wherein the hybridization sequence further includes a poly (T) tail complementarily binding to the poly (A) tail of the mRNA at the 3′ end.

4. The nucleic acid construct of claim 3, wherein the poly (T) tail has a length of 2 to 6 nt.

5. The nucleic acid construct of claim 1, wherein the nucleic acid construct simultaneously induces expression of a target protein encoded by the mRNA and an expression inhibition of a target gene.

6. The nucleic acid construct of claim 1, further comprising:
a first linker sequence for an additional siRNA sense or antisense sequence at the 3′ end of the first siRNA sense or antisense sequence.

7. The nucleic acid construct of claim 6, wherein the first linker sequence is a poly (U) tail.

8. The nucleic acid construct of claim 6, further comprising:
an additional siRNA sense or antisense sequence at the 3′ end of the first linker sequence.

9. The nucleic acid construct of claim 8, further comprising:
a hybridization sequence; and a second linker sequence, wherein the hybridization sequence complementarily binds to the first linker sequence; and the additional siRNA sense or antisense sequence.

10. The nucleic acid construct of claim 9, wherein the hybridization sequence is a second siRNA antisense or sense sequence complementarily binding to the additional siRNA sequence; or a DNA antisense or sense sequence complementarily binding to the additional siRNA sequence.

11. The nucleic acid construct of claim 9, wherein the second linker sequence is a poly (A) tail complementarily binding to the first linker sequence.

12. The nucleic acid construct of claim 1, further comprising:
a plurality of repeating units comprising the following (a) and (b) at the 3′ end of the first siRNA sense or antisense sequence:
(a) a first linker sequence for an additional siRNA sense or antisense sequence; and an additional siRNA sense or antisense sequence; and
(b) a hybridization sequence; and a second linker sequence, wherein the hybridization sequence complementarily binds to the first linker sequence; and the additional siRNA sense or antisense sequence.

13-17. (canceled)

18. A cancer vaccine comprising the nucleic acid construct of claim 1.

19. The cancer vaccine of claim 18, further comprising:
dendritic cells.

20. A method for preparing a nucleic acid construct comprising mRNA and siRNA, comprising:
1) preparing a DNA template into which a poly (T) tail and an siRNA antisense or sense sequence located sequentially from a 5′ direction to a 3′ direction at a 3′ end are introduced;
2) obtaining mRNA including the siRNA sequence at the 3′ end by treating the DNA template with a reaction solution including RNA polymerase; and
3) annealing siRNA having oligo (dT) linked at the 3′ end to the mRNA product.

21-23. (canceled)

24. An immunotherapy method comprising administering the nucleic acid construct of claim 1 to a subject in need thereof.

25. An immunotherapy method of claim 24, wherein the immunotherapy is cancer immunotherapy.

26. An immunotherapy method of claim 24, wherein the cancer is at least one kind selected from the group consisting of brain tumor, benign astrocytoma, malignant astrocytoma, pituitary adenoma, meningioma, brain lymphoma, oligodendroglioma, craniopharyngioma, ependymoma, brainstem tumor, head and neck tumor, laryngeal cancer, oropharyngeal cancer, nasal/sinus cancer, nasopharyngeal cancer, salivary gland cancer, hypopharyngeal cancer, thyroid cancer, chest tumor, small cell lung cancer, non-small cell lung cancer, thymus cancer, mediastinal tumor, esophageal cancer, breast cancer, male breast cancer, abdominal tumor, stomach cancer, liver cancer, gallbladder cancer, biliary tract cancer, pancreatic cancer, small intestine cancer, colorectal cancer, anal cancer, bladder cancer, kidney cancer, male genital tumor, penile cancer, urethral cancer, prostate cancer, female genital tumor, cervical cancer, endometrial cancer, ovarian cancer, uterine sarcoma, vaginal cancer, female external genital cancer, female urethral cancer, skin cancer, myeloma, leukemia and malignant lymphoma.
